(19) United States
(12) Patent Application Publication
(10) Pub. No.: US 2022/0304453 A1
(43) Pub. Date: Sep. 29, 2022

(54) INFORMATION PROCESSING DEVICE, COMPUTER READABLE MEDIA, NAIL PRINTING SYSTEM AND NAIL DESIGN SELECTION METHOD

(71) Applicant: CASIO COMPUTER CO., LTD., Tokyo (JP)

(72) Inventors: Toshihiko OTSUKA, Ome-shi, Tokyo (JP); Takahiro TOMIDA, Hamura-shi, Tokyo (JP)

(73) Assignee: CASIO COMPUTER CO., LTD., Tokyo (JP)

(21) Appl. No.: 17/701,190

(22) Filed: Mar. 22, 2022

(30) Foreign Application Priority Data
Mar. 24, 2021 (JP) 2021-050213
Mar. 24, 2021 (JP) 2021-050214

(51) Int. Cl. A45D 44/00; A61B 5/026; A61B 5/021; A61B 5/024; A45D 29/00

(52) U.S. Cl. CPC A45D 44/005; A61B 5/0261; A61B 5/021; A61B 5/02416; A45D 29/00; A61B 2503/12; A45D 2044/007; A45D 2029/005

(57) ABSTRACT

The present invention aims to provide an information processing device, computer readable media, a nail printing system and a nail design selection method which make it possible to present nail designs which are more suitable for a psychosomatic state of a user.

A print device includes a control unit which acquires biological information which relates to a blood flow of the user and determines nail designs to be presented to the user on the basis of the acquired biological information.

### CROSS REFERENCE TO RELATED APPLICATIONS

This application is based upon and claims the benefit of multiple priorities under 35 USC 119 of Japanese Patent Application No. 2021-050213 and Japanese Patent Application No. 2021-050214 both filed on Mar. 24, 2021 the entire disclosure of which, including the description, claims, drawings, and abstract, is incorporated herein by reference in its entirety.

### BACKGROUND OF THE INVENTION

### 1. Field of the Invention

The present invention relates to an information processing device, computer readable media, a nail printing system and a nail design selection method.

### 2. Description of the Related Art

Today, a nail printer which is configured to print a nail design of a color, a pattern and so forth that a user selects on a user's finger nail is known. This kind of technology is described in, for example, Japanese Patent Application Laid Open No. 2017-18589. In addition, in Japanese Patent Application Laid Open No. 2020-62529, there is such a description that a video signal of an object person is acquired as an image and a luminance or a video pulse wave is extracted on the basis of the acquired image.

### BRIEF SUMMARY OF THE INVENTION

According to one aspect of the present invention, there is provided an information processing device which includes a process unit which acquires biological information which relates to a blood flow of a user and determines nail designs on the basis of the acquired biological information.

According to one aspect of the present invention, there is also provided a nail printing system which includes a print device which has a first communication unit which communicates with the outside and a print unit which performs printing on a nail of a user and an information processing device which has a second communication unit which communicates with the print device and a process unit which determines biological information of the user on the basis of information which relates to a video of at least a part of the user's body and determines nail designs on the basis of the determined biological information, in which the print unit performs printing on the user's nail on the basis of the nail designs that the first communication unit receives from the second communication unit.

According to one aspect of the present invention, there is further provided computer readable media, making a computer execute a processing function of acquiring biological information which relates to a blood flow of a user and a processing function of determining nail designs to be presented to the user on the basis of the acquired biological information.

According to one aspect of the present invention, there is further provided a nail design selection method that an information processing device executes, including acquiring biological information which relates to a blood flow of a user and determining nail designs to be presented to the user on the basis of the acquired biological information.

### BRIEF DESCRIPTION OF THE SEVERAL VIEWS OF THE DRAWING

FIG. 1 is a configuration diagram illustrating one example of a configuration of a nail printing system pertaining to one embodiment of the present invention.

FIG. 2 is a block diagram illustrating one example of a hardware configuration of a print device pertaining to one embodiment of the present invention.

FIG. 3 is a configuration diagram illustrating one example of a configuration of a print unit pertaining to one embodiment of the present invention.

FIG. 4 is a function block diagram illustrating one example of a functional configuration for executing print processing in functional configurations of the print device pertaining to one embodiment of the present invention.

FIG. 5 is a block diagram illustrating one example of a hardware configuration of a user terminal pertaining to one embodiment of the present invention.

FIG. 6 is a function block diagram illustrating one example of a functional configuration for executing print processing in functional configurations of the user terminal pertaining to one embodiment of the present invention executes.

FIG. 7 is a schematic diagram illustrating one example of explanation of a measurement region which is set in a skin region of a finger.

FIG. 8 is a schematic diagram illustrating one example of explanation of acquisition of pulse wave information from a luminance value of pixels in the measurement region.

FIG. 9 is a schematic diagram illustrating one example of explanation of calculation of biological information from the pulse wave information.

FIG. 10 is a table that the biological information which is calculatable, decision threshold values which correspond to respective pieces of the biological information, selection of nail designs to be presented to a user and results of estimation of psychosomatic states are listed.

FIG. 11 is a schematic diagram illustrating one example of a selection screen of nail designs which are presented to the user.

FIG. 12 is a graph illustrating one example of a Lorenz plot which is calculated from the pulse wave information.

FIG. 13 is part of a flowchart explaining one example of a flow of processing that the print device pertaining to one embodiment of the present invention executes.

FIG. 14 is part of the flowchart explaining one example of the flow of processing that the print device pertaining to one embodiment of the present invention executes.

FIG. 15 is a block diagram illustrating one example of a hardware configuration of the print device pertaining to a first modified example of the present invention.

FIG. 16 is a configuration diagram illustrating one example of a configuration of a print unit pertaining to the first modified example of the present invention.

FIG. 17 is a functional block diagram illustrating one example of a functional configuration for execution of print processing in functional configurations of the print device pertaining to the first modified example of the present invention.

FIG. 18 is explanatory diagrams illustrating one example of a difference between pulse wave measurement results which is induced depending on whether a finger is pressed with a cuff.

FIG. 19 is a flowchart illustrating one example of part of a flow of processing that the print device pertaining to the first modified example of the present invention executes.

FIG. 20 is a flowchart illustrating one example of part of a flow of processing that the print device pertaining to a second modified example of the present invention executes.

FIG. 21 is a schematic diagram illustrating one example of a configuration of a nail printing system pertaining to a third modified example of the present invention.

### DETAILED DESCRIPTION OF THE INVENTION

In the following, one embodiment of the present invention will be described by using the drawings.

### [Outline of Embodiment]

### <Nail Printing System>

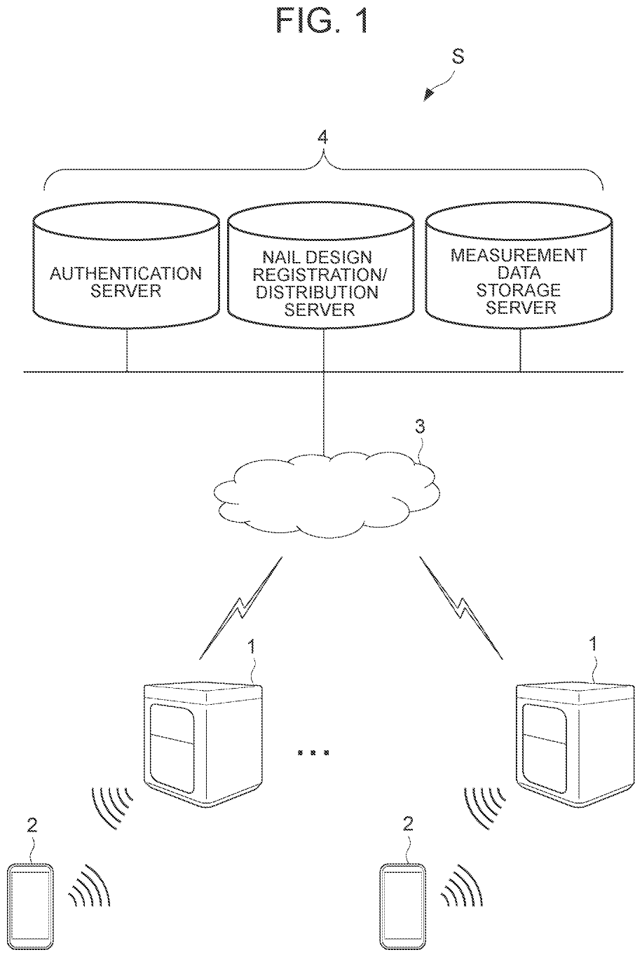

FIG. 1 is a configuration diagram illustrating one example of the entire configuration of a nail printing system S that a print device 1 pertaining to one embodiment of the present invention is included. As illustrated in FIG. 1, the nail printing system S includes the plurality of print devices 1, user terminals 2 which are tablet terminals which are prepared in one to one correspondence with the print devices 1, a network 3 and a server group 4.

The print device 1 is a nail printer which prints a nail design which is created by using colors, pictorial patterns, patterns of various forms, tones, characters and so forth on a user's nail. There is no specific limitation on the number of the print devices 1 to be installed and n (n is an arbitrary natural number) print devices 1 may be included in the nail printing system S. Incidentally, in the following description, in a case where description is made with no specific distinction of the n print devices 1, the print device will be simply referred to as “the print device 1” by omitting an alphabet which is appended to the symbol “1”.

The print device 1 pertaining to one embodiment measures a blood flow fluctuation of the user from a video and makes the user select a nail design on the basis of a result of measurement. The print device 1 prints the nail design that the user selects on the user's nail. In addition, the print device 1 is electrically connected with the user terminal 2 via wired communications or wireless communications and transmits and receives various kinds of information to and from the user terminal 2. In addition, the user terminal 2 is connected to each server which is included in the server group 4 over the network 3 to be mutually communicable.

The network 3 is realized by any one of, for example, the Internet, a LAN (Local Area Network) and a mobile telephone network or by a combination of these networks.

Various kinds of servers which cooperate with the print devices 1 are included in the server group 4. For example, an authentication server which is adapted to authenticate the user of each print device 1 is included in the server group 4. In addition, for example, a nail design registration/distribution server that nail design data which is to be downloaded to the user terminals 2 is registered is included in the server group 4. Further, a measurement data storage server which stores profile information of the users is included in the server group 4. Setting information which relates to each user and information which relates to a usage history that each user utilizes each print device 1 and so forth are included in profile information of each user.

The nail design is applied to the user's finger nail. The nail design which is created by using, for example, the colors, the pictorial patterns, the patterns of various forms, the tones, the characters and so forth is applied to the user's finger nail. The nail design is not only artistic but also has such psychological effects that a stress on a person who is applied with the nail design is relieved and motivation of that person is increased with the aid of the impression and so forth of the nail design.

Incidentally, the nail printing system S which is illustrated in FIG. 1 is merely one example and servers which have other functions may be included in the server group 4. In addition, the plurality of servers which is included in the server group 4 may be realized by mutually different server devices and may be also realized by a single server device.

### <Print Device>

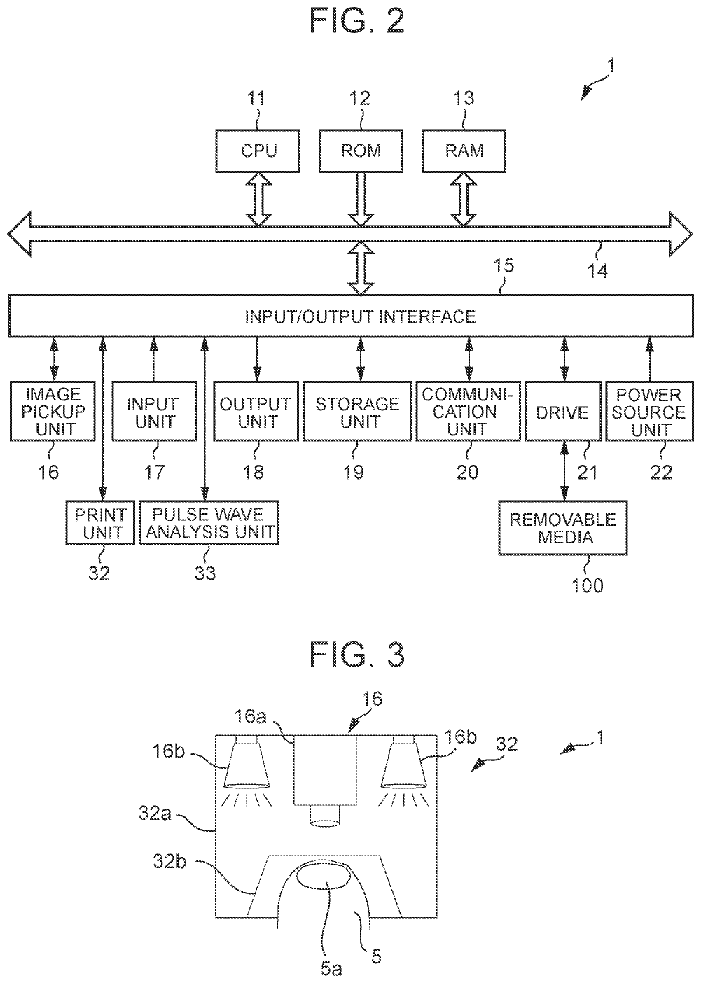

FIG. 2 is a block diagram illustrating one example of a hardware configuration of the print device 1 pertaining to one embodiment of the present invention. As illustrated in FIG. 2, the print device 1 includes a CPU (Central Processing Unit) 11 which is a processor, a ROM (Read Only Memory) 12, a RAM (Random Access Memory) 13, a bus 14, an input/output interface 15, an image pickup unit 16, an input unit 17, an output unit 18, a storage unit 19, a communication unit 20, a drive 21, a power source unit 22, a print unit 32 and a pulse wave analysis unit 33.

The CPU 11 executes various kinds of processing in accordance with a program which is recorded in the ROM 12 or a program which is loaded from the storage unit 19 to the RAM 13.

In addition, data and so forth which are necessary for the CPU 11 in order to execute the various kinds of processing are appropriately stored in the RAM 13.

The CPU 11, the ROM 12 and the RAM 13 are mutually connected via the bus 14. In addition, the input/output interface 15 is also connected to the bus 14. The image pickup unit 16, the input unit 17, the output unit 18, the storage unit 19, the communication unit 20, the drive 21 the power source unit 22, the print unit 32 and the pulse wave analysis unit 33 are connected to the input/output interface 15.

As illustrated in FIG. 3, the image pickup unit 16 has a camera 16a which includes an optical lens section and an image sensor and a lamp 16b which is installed in a housing 32a which will be described later and illuminates the inside of the housing 32a. The optical lens section is configured by a lens which condenses light such as, for example, a focus lens, a zoom lens and so forth in order to pick up an image of a subject. The focus lens makes an image of the subject form on a light receiving surface of the image sensor. The zoom lens freely changes a focal length within a fixed range. In addition, a peripheral circuit which adjusts setting parameters such as a focal point, exposure, white balance and so forth is installed in the image pickup unit 16 as necessary.

The image sensor is configured by a photoelectric conversion element, an AFE (Analog Front End) and so forth. The photoelectric conversion element is configured by, for example, a CMOS (Complementary Metal Oxide Semiconductor) type photoelectric conversion element and so forth. A subject image is made incident on the photoelectric conversion element through the optical lens section. Then, the photoelectric conversion element performs photoelectric conversion on the subject image (picks up the image) and accumulates image signals for a fixed time, and then sequentially supplies the accumulated image signals to the AFE as analog signals. The AFE executes various kinds of signal processing such as A/D (Analog/Digital) conversion processing and so forth on these analog image signals. Digital signals are generated by execution of the various kinds of signal processing and are output from the image pickup unit 16 as output signals. The output signals which are output from the image pickup unit 16 in this way are appropriately supplied to the CPU 11 and so forth. The lamp 16b is configured by, for example, an LED (Light Emitting Diode).

The input unit 17 is configured by various buttons, a microphone and so forth and inputs various kinds of information in accordance with an instruction operation which is performed by the user and voice instructions that the user issues.

The output unit 18 is configured by a liquid crystal display and so forth and outputs images and videos which correspond to image data and video data that the CPU 11 outputs.

The storage unit 19 is configured by a semiconductor memory such as a DRAM (Dynamic Random Access Memory) and so forth and stores various kinds of data.

The communication unit 20 performs communication control which is necessary for communication that the CPU 11 makes with other devices (for example, the respective servers which are included in the server group 4) over the network 3.

The drive 21 is equipped with an interface which makes it possible to attach removable media 100 to the drive 21. The removable media 100 which are configured by a magnetic disk, an optical disk, a magneto-optical disk, a semiconductor memory and so forth are appropriately attached to the drive 21. Various kinds of programs and various kinds of data such as image data, video data and so forth may be stored into the removable media 100. Further, the various kinds of programs and the various kinds of data such as the image data, the video data and so forth which are read out of the removable media 100 via the drive 21 may be installed into the storage unit 19 as necessary.

The power supply unit 22 is configured to allow electric power supply to respective units of the print device 1 by being connected to an external power source.

The print unit 32 prints a nail design on the user's nail 5a. As illustrated in FIG. 3, the print unit 32 has a finger placement stand 32b for placing a finger 5 of the user, a not illustrated sensor which detects the finger 5 which is placed on the finger placement stand 32b, a not illustrated nail design print section which prints the nail design on the nail 5a and a housing 32a which covers the finger placement stand 32b, the sensor and the nail design print section. That is, in the print device 1 pertaining to one embodiment, the nail design print section prints the nail design on the nail 5a of the finger 5 which is placed on the finger placement stand 32b.

Incidentally, the print device 1 pertaining to one embodiment recognizes the finger 5 which is placed on the finger placement stand 32b by the sensor. However, the print device 1 may recognize the finger 5 on the basis of the video which is picked up by the camera 16a of the image pickup unit 16, in place of the sensor. For example, the print device 1 may recognize the finger 5 by detecting skin color of the finger 5 from the picked-up video.

A pulse wave analysis unit 33 has a pulse wave analysis circuit which calculates pulse wave information by analyzing a video of the skin and so forth of the user. For example, when the video that the image pickup unit 16 picked up is input, the pulse wave analysis unit 33 calculates various kinds of biological information which will be described later via the pulse wave analysis circuit. In one embodiment, analysis of the video is performed by a video processing section 111 of a control unit 110 which will be described later and thereby the pulse wave information and the various biological information are calculated. Alternatively, the pulse wave analysis unit 33 may calculate the pulse wave information and the various kinds of biological information respectively.

In addition, the print device 1 may further include other hardware in addition to the above-described hardware. For example, the print device 1 may further include an output unit which is configured by a lamp, a loudspeaker, and/or a motor for vibration and/or so forth and outputs light, voice and/or a vibration signal and/or so forth.

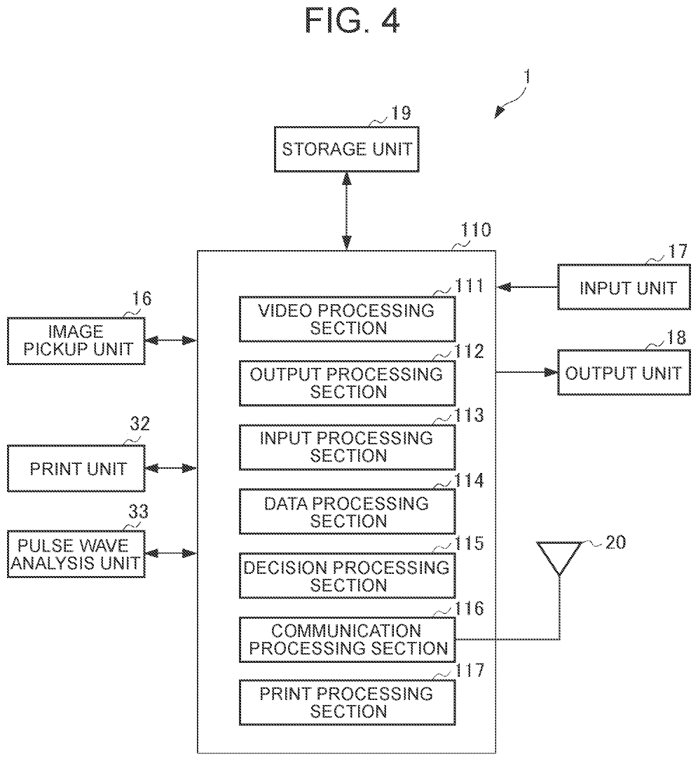

FIG. 4 is a functional block diagram illustrating one example of a functional configuration for execution of print processing in functional configurations of the print device 1 in FIG. 2. The print processing means a series of processing which includes display processing that the print device 1 displays a measurement result on the basis of a change in each piece of the biological information that the print device 1 acquires from the user.

First, the storage unit 19 which stores various kinds of information will be described. Various kinds of data on guidance in screen display processing, information which is used for performing measurement, information which is used for displaying the measurement result, information which indicates the measurement result, information which relates to the nail designs, a history that each nail design is printed, information which relates to the print processing and so forth are stored in the storage unit 19. Incidentally, although the above-described various kinds of data may be stored into only the storage unit 19, these various kinds of data may be also appropriately stored into the removable media 100 by the drive 21. Further, each piece of information may be appropriately stored into a measurement data storage server and so forth which are included in the server group 4.

Next, each functional block which executes the print processing will be described. As illustrated in FIG. 4, the control unit 110 pertaining to one embodiment has a video processing section (a video processing function) 111, an output processing section (an output processing function) 112, an input processing section (an input processing function) 113, a data processing section (a data processing function) 114, a decision processing section (a decision processing function) 115, a communication processing section (a communication processing function) 116 and a print processing section (a print processing function) 117.

The video processing section 111 analyzes the video which contains the user as the subject that the image pickup unit 16 picks up and thereby acquires the information which relates to the user (in the following, will be referred to as “subject information”). The subject information is biological information (also called vital data as the case may be) and so forth which indicate, for example, a color of each portion of the finger 5 and so forth in the user video and a state of the user. Measurement is performed by analyzing the information (the video) that the image pickup unit 16 acquires and therefore it is possible to sequentially acquire the biological information with no contact with the user. Concrete analysis of the video data by the video processing section 111 will be described later. In addition, when outputting the acquired biological information, the video processing section 111 outputs that acquired biological information at least to the storage unit 19. Thereby, the history of the acquired biological information is stored into the storage unit 19.

The output processing section 112 executes control processing for displaying a moving image as a video to be displayed on the screen of the output unit 18. Thereby, it becomes possible to dynamically visualize and thereby intelligibly display a progress status and so forth of pulse wave measurement. In addition, the output processing section 112 also executes synthetic processing of synthesizing a guide image, measurement data, the nail designs and so forth and other processing. For example, the output processing section 112 executes control processing for displaying such a synthetic video that the user's finger 5 and images (a measurement result table, a graph and so forth) which indicate the measurement result are synthesized as that which is illustrated in FIG. 9 and a preview video that the user's finger 5 and a nail design image are synthesized.

In addition, the input processing section 113 executes processing of accepting an operation which is input into the input unit 17 by the user. For example, the input processing section 113 accepts an operation of starting execution of the print processing which is input into the input unit 17 by the user and inputs the accepted operation into the print processing section 117.

The data processing section 114 executes processing for printing the nail design which is based on the biological information. For example, the data processing section 114 acquires the biological information which is calculated by the video processing section 111. The data processing section 114 outputs the acquired biological information to the communication processing section 116 and makes the communication processing section 116 transmit the acquired biological information to the user terminal 2 via the communication unit 20. In addition, the data processing section 114 acquires information which relates to the nail design which is transmitted from the user terminal 2 and outputs a print command signal to the print unit 32.

The decision processing section 115 makes various decisions in the print processing on the basis of various threshold values which are stored in the storage unit 19. For example, the decision processing section 115 comparers a value of each piece of the biological information which is calculated by the video processing section 111 with each predetermined threshold value and decides whether each value is more than or less than each threshold value and outputs each result of decision to the data processing section 114.

The communication processing section 116 executes processing for communicating with external equipment. For example, the communication processing section 116 communicates with the authentication server which is included in the server group 4. Thereby, the user to whom the printing processing is to be executed is authenticated. In addition, the communication processing section 116 communicates with the measurement data storage server which is included in the server group 4 and thereby updates profile information of the user which is used in execution of the print processing.

The print processing section 117 executes processing of controlling an operation of the print unit 32 of the print device 1. For example, in a case where the print processing section 117 receives a print start command from the control unit 110, the print processing section 117 makes the print unit 32 print the nail design on the user's nail 5a.

### <User Terminal>

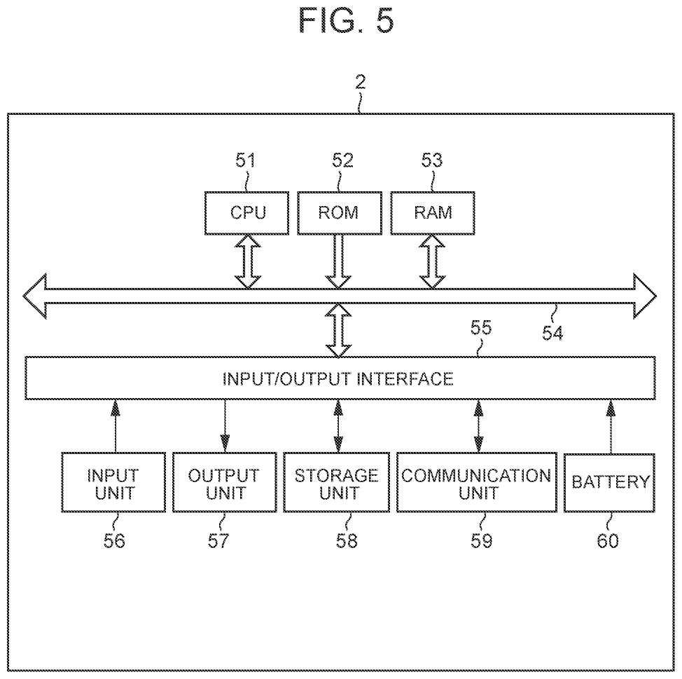

Next, one example of the user terminal 2 will be described by using FIG. 5. FIG. 5 is a block diagram illustrating one example of a hardware configuration of the user terminal 2 pertaining to one embodiment of the present invention.

The user terminal 2 pertaining to one embodiment is a computer which includes a CPU 51, a ROM 52, a RAM 53, a bus 54, an input/output interface 55, an input unit 56, an output unit 57, a storage unit 58, a communication unit 59 and a battery 60. Incidentally, there are cases where as for a configuration which is common to or the same as the already described configuration, the same term is assigned to that configuration and detailed description thereof is omitted.

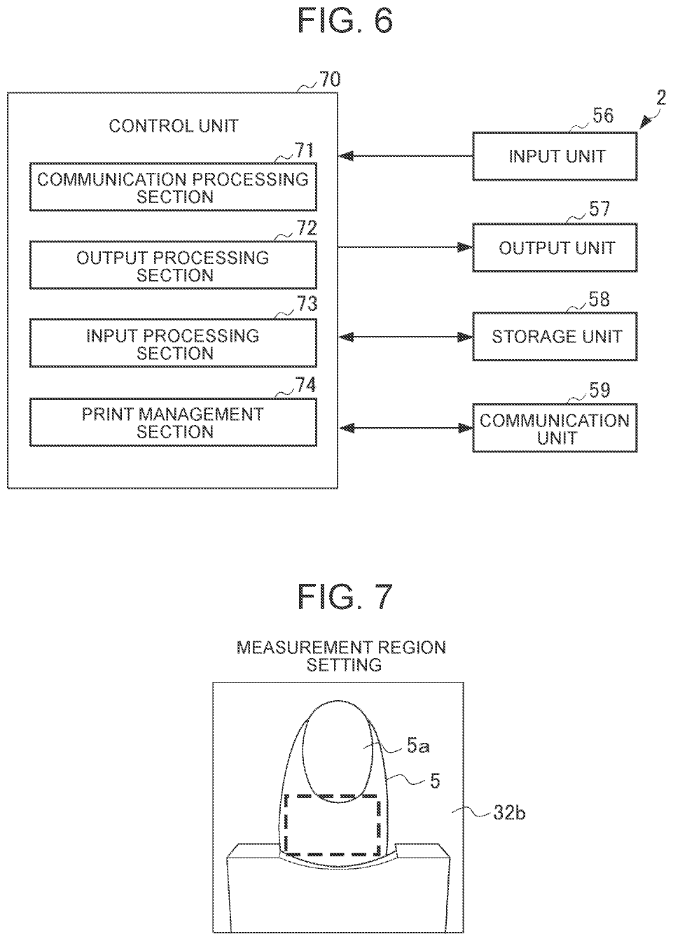

Next, a functional configuration of the user terminal 2 will be described. FIG. 6 is a functional block diagram illustrating one example of part of the functional configuration of the user terminal 2 pertaining to one embodiment.

A control unit 70 which performs various controlling operations of the user terminal 2 is realized by the CPU 51 which executes arithmetic processing. The control unit 70 pertaining to one embodiment has a communication processing section (a communication processing function) 71, an output processing section (an output processing function) 72, an input processing section (an input processing function) 73 and a print management section (a print management function) 74.

The communication processing section 71 executes processing of exchanging various kinds of information with the server group 4 via the communication unit 59. In addition, the communication processing section 71 executes processing of exchanging various kinds of information with the print device 1 via the communication unit 59.

The output processing section 72 executes processing for displaying an image on a screen of the output unit 57. For example, in a case where the communication processing section 71 acquires the biological information which is transmitted from the print device 1, the output processing section 72 executes processing for displaying the biological information on the screen of the output unit 57 in accordance with a command from the communication processing section 71. In addition, in a case where the communication processing section 71 receives nail design selection information which is transmitted from the nail design registration/distribution server (will be described later) in the server group 4, the output processing section 72 executes processing of displaying nail design selection information on the screen of the output unit 57 in accordance with a command from the communication processing section 71.

The input processing section 73 executes processing of accepting a user's operation which is input into the input unit 56. For example, on a touch panel, the input processing section 73 accepts information which is displayed on the output unit 57 as input information and transmits a command for making the output processing section 72 execute processing of switching information to be displayed on the screen of the output unit 57 and so forth to the output processing section 72.

The print management section 74 transmits and receives information which relates to the print processing to and from the print device 1 via the communication unit 59. For example, in a case of accepting user's input for start of printing, the print management section 74 outputs a command to start printing to the communication processing section 71 and thereby makes the print device 1 start execution of the print processing via the communication unit 59.

### [Analysis of Video Data]

Next, analysis of the video data in the print device 1 will be described by using FIG. 7 to FIG. 9.

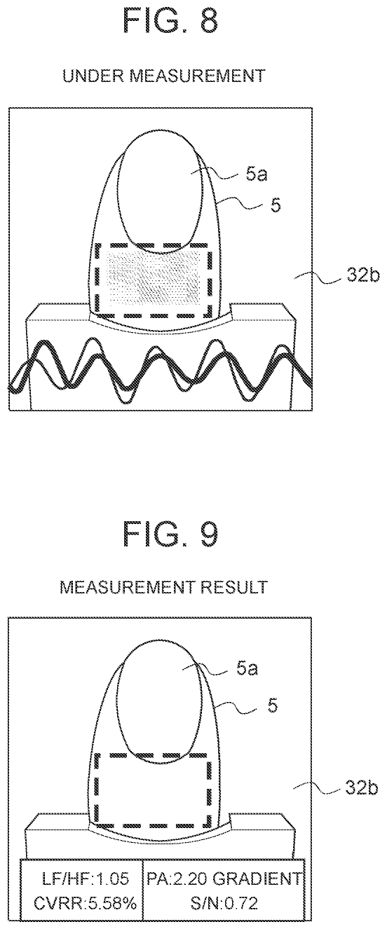

FIG. 7 is a schematic diagram illustrating one example of a state where the finger 5 which is placed on the finger placement stand 32b in FIG. 3 and a region of interest ROI (Region Of Interest) which is set as a measurement region in a skin region of the finger 5 are displayed. FIG. 8 is a schematic diagram illustrating one example of explanation that a luminance value of pixels in the region of interest ROI is calculated and then a waveform of an acquired pulse wave is displayed. A fine waveform indicates an original waveform of the pulse wave and a bold waveform indicates a waveform of the pulse wave which is obtained after passing through a band pass filter. FIG. 9 is a schematic diagram illustrating one example of explanation that the biological information is calculated from the measured pulse wave information. First, acquisition of video data which is analyzed by the video processing section 111 will be described by using FIG. 7.

The video processing section 111 executes processing which relates to pattern matching of an outline and a part of the finger 5 and tracking of skin color identification and so forth and thereby recognizes the outline and a position of the finger 5 and a skin region and detects a predetermined part region of the finger 5. The video processing section 111 detects the outline and the position of the finger 5 from, for example, a user video in the video and thereby the region of the finger 5 is automatically recognized. Then, the video processing section 111 detects states of the skin color and so forth in the detected finger part region. In addition, as illustrated in FIG. 7, the video processing section 111 sets the region of interest ROI which is indicated with a dashed-line frame on the root side of the nail 5a of the finger 5.

Incidentally, in one embodiment, the region of interest ROI is set on a skin part other than the nail 5a. This is because white base cote is applied to the nail 5a for position recognition and measurement of the pulse wave is difficult on the base-cote-applied part. In a case where the base cote is not applied to the nail 5a, the region of interest ROI may be set on the nail 5a. In addition, in one embodiment, the region of interest ROI is set within a predetermined range. However, the region of interest ROI may not necessarily be set within the predetermined range. For example, the control unit 110 may automatically recognize a skin region from the skin color in the video and may automatically set a range that an area of the region of interest ROI reaches a maximum.

The video processing section 111 acquires the biological information which relates to the blood flow such as the pulse, the pulse wave and so forth by utilizing such a property that hemoglobin in the blood absorbs green light well in extraction of a pulse wave from the video. It is generally said that the wavelength of the green signal ranges, for example, from 495 nm to 570 nm and the absorption coefficient of the hemoglobin is increased in a range, for example, from 500 nm to 600 nm. When the blood flow velocity is increased, the volume of the blood is increased on the skin surface and the amount of hemoglobin per unit time is increased. Therefore, a larger number of green signals is absorbed by the hemoglobin in comparison with the number of green signals obtained before the blood flow velocity is increased. As a result, the luminance of the green signal which is detected when the blood flow velocity is increased is lowered. Incidentally, in a case where the image pickup element of the image pickup unit 16 converts luminous intensity to luminance, an RGB filter is arranged in front of the image pickup element so as to calculate luminance values of R, G and B pixels respectively. In this case, the light intensity of light which passes through a green filter becomes a luminance value. Even in a case where the sensitivity of the image pickup element is not flat relative to a wavelength, it is possible to narrow down a wavelength band to some extent by the above-described filters and therefore it is possible to accurately detect the green signal.

As illustrated in FIG. 8, the video processing section 111 acquires pulse wave information on the basis of luminance information which is contained in video information on a body in the video in the region of interest ROI. More specifically, the video processing section 111 acquires the luminance of the green signal in unit time and then acquires the pulse wave information from a time-dependent change in luminance of the green signal. Incidentally, the unit time is, for example, a frame rate of a moving image and it is possible to acquire the luminance of the signal per temporally sequential images which configure the video.

Further, the video processing section 111 may also acquire the luminance of the red signal in unit time in the same way and may calculate the pulse wave information by subtracting the luminance of the red signal from the luminance of the green signal which is acquired in unit time. It is possible for the video processing section 111 to acquire the pulse wave information from the time-dependent change in luminance that the luminance of the red signal is subtracted from the luminance of the green signal and to more increase accuracy of the pulse wave information. In addition, in one embodiment, operations ranging from an operation of picking up the image of a target part of a user's body by the image pickup unit 16 to an operation of acquiring the luminance of the green signal are described as operations for the pulse wave measurement.

In addition, in the pulse wave information, an amplitude PA (Pulse Amplitude) is contained. In FIG. 8, a thick-line waveform is a waveform out of which an amplitude component is taken by passing the original waveform of the pulse wave through a bandpass filter. The pulse wave which is analyzed from the video exhibits a waveform which is periodically generated within a fixed range of the amplitude PA. The amplitude PA of this pulse wave means a difference between mutually adjacent maximum value and minimum value of the pulse wave waveform. That is, the amplitude PA means a difference between a maximum value and a minimum value of the luminance of the green signal.

Incidentally, it is preferable that a range which is set for acquisition of the amplitude PA be a region that the amplitude is stable with no abnormal value. For example, in a case where the abnormal value which exceeds a threshold value which is set in advance is detected and so forth, the pulse wave information is acquired so as to exclude the abnormal value. Alternatively, a message that appropriate acquisition of the video is difficult when picking-up the image may be displayed and appropriate pulse wave information may be acquired by picking up the image again. Also alternatively, a pulse wave which is obtained after a predetermined time has elapsed from start of image-picking-up may be used for calculation of the amplitude. Still alternatively, the amplitude may be calculated by removing the abnormal value from values of the pulse wave which are acquired in a predetermined time. It is possible to apply various methods to calculation of the amplitude in this way.

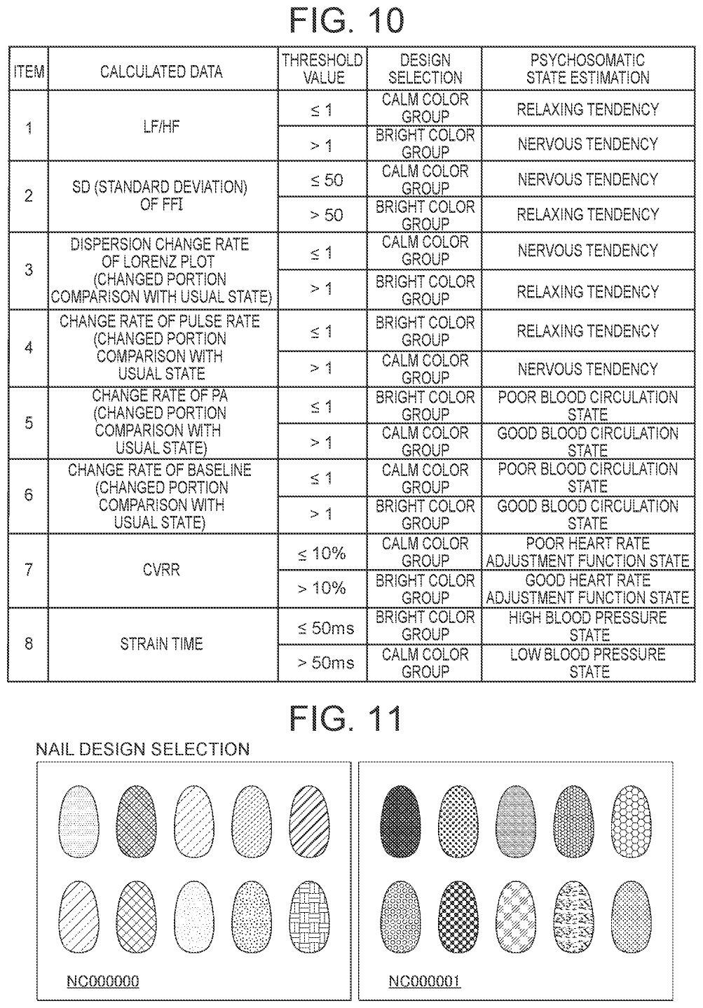

Further, it becomes possible for the video processing section 111 pertaining to one embodiment to calculate various kinds of biological information which will be described later by performing frequency analysis, time domain analysis and so forth on the pulse wave which is acquired by the video analysis as illustrated in FIG. 9. In one embodiment, as illustrated in FIG. 10, it is possible to calculate psychosomatic biological information such as LF/HF (Low Frequency/High Frequency), SD (Standard Deviation) of FFI (Foot to Foot Interval), a dispersion change rate of Lorenz plot, a change rate of pulse rate, a change rate of PA, a change rate of baseline, CVRR (Coefficient of Variation of R-R intervals), a strain time and so forth. Details of each piece of biological information will be described later.

In addition, it is possible for the print device 1 pertaining to one embodiment to estimate four psychosomatic states as illustrated in FIG. 10 by setting various kinds of biological information as indexes and comparing these indexes with threshold values in FIG. 10 respectively. FIG. 10 is a table that the biological information calculation of which is possible for the print device 1, decision threshold values which correspond to the various kinds of biological information respectively, selection of nail designs which correspond to each result of decision of the psychosomatic state of the user and are to be presented to the user and psychosomatic state estimation results are put together.

For example, as illustrated in FIG. 10, it is possible to estimate whether the user's psychosomatic state tends to get nervous or relaxed from LF/HF, SD (Standard Deviation) of FFI, the dispersion change rate of Lorenz plot and the change rate of pulse rate. In addition, it is possible to estimate whether a user's blood circulation is good or bad from the change rate of PA and the change rate of baseline. In addition, it is possible to estimate whether a user's heart rate adjustment function is good or bad and whether a user's blood pressure is high or low from CVRR and the strain time respectively. Incidentally, the threshold values which are indicated in FIG. 10 are merely examples and it is possible to appropriately change numerical values and conditions.

In addition, it is possible for the print device 1 pertaining to one embodiment to present nail designs as illustrated in FIG. 11 on the basis of the estimated user's psychosomatic state and in accordance with three guidelines. As the three guidelines, a guideline that the user's psychosomatic state is expressed by a nail design, a guideline which indicates a nail design which keeps balance of the user's psychosomatic state and a guideline which indicates a nail design which has an effect of maintaining user's health are available.

For example, in the guideline that the psychosomatic state of the user is expressed by nail designs, in a case where it is estimated that the user's psychosomatic state tends to be nervous, the print device 1 presents bright-color nail designs which express a nervous state to the user. In addition, in a case where it is estimated that the user's psychosomatic state is a relaxed state, the print device 1 presents calm-color nail designs to the user. Incidentally, in one embodiment, although the nail designs are described mainly in relation to the colors thereof, the pictorial patterns, the patterns of various shapes and decorations may be also changed in accordance with the estimated psychosomatic state of the user.

In addition, in the guideline which indicates the nail designs which keep balance of the user's psychosomatic state, in a case where it is estimated that the user's psychosomatic state tends to be nervous, the print device 1 presents calm-color nail designs which relieve the nervous state to the user. In addition, in a case where it is estimated that the user's psychosomatic state tends to be relaxed, the print device 1 presents bright-color nail designs which increase the user's motivation to the user.

In addition, in the guideline which indicates the nail design which has the effect of maintaining the user's health, in a case where it is estimated that the user exhibits a high blood pressure fluctuation state, the print device 1 presents calm-color nail designs to the user. In addition, in a case where it is estimated that the user exhibits a low blood pressure fluctuation state, the print device 1 presents bright-color nail designs to the user.

In addition, it is possible for the print device 1 to estimate the user's psychosomatic state on the basis of one index and to determine the nail designs to be presented to the user. Alternatively, the print device 1 may determine the nail designs by estimating the user's psychosomatic state by combining a plurality of indexes together. For example, the print device 1 may estimate the user's psychosomatic state in relation to each of four items, that is, whether the user exhibits the relaxing tendency or the nervous tendency, whether the user's blood circulation is good or bad, whether the user's pulse adjustment function is good or bad and whether the user's blood pressure is high or low and then may decide results of estimation of the four items by majority vote. In addition, the print device 1 may select any one of the items which is used for decision making, not by majority vote.

In addition, the nail designs may be decided by selecting one item which is suited for processing capability (the resource) of a system to be implemented. For example, in a print device which is low in processing capability, an index which is large in load which is exerted when calculating the index may not be selected. Incidentally, although the indexes to be used for decision are set in advance in one embodiment, such a setting that the index to be used for decision is selected by the user may be also made.

In one embodiment, it is possible for the user to set in advance as to which nail designs are presented in accordance with which guideline. In print processing pertaining to one embodiment which will be described later, the guideline that the user's psychosomatic state is expressed by the nail design is selected. Incidentally, the guidelines for nail design selection which is based on the user's psychosomatic state are not limited to the above-described three guidelines, one guideline may be selected from the three guidelines and a guideline other than the above-described three guidelines may be selected. In addition, the guidelines for nail design selection which are set in accordance with the user's psychosomatic state may be fixed, instead of being selected by the user.

Next, details of each piece of the biological information will be described. First, LF/HF is the index which indicates a fluctuation in interval between pulse-wave pulses and indicates an autonomic tendency. In addition, LF/HF is calculated by frequency analysis and so forth. The larger the numerical value of LF/HF is, the more it becomes possible for the print device 1 to decide that the user tends to be nervous. In addition, the smaller the LF/HF numerical value is, the more it becomes possible for the print device 1 to decide that the user tends to be relaxed.

Next, the standard deviation of FFI will be described. FFI is the index which indicates a peak-to-peak time interval of the bottom of a pulse wave waveform. The standard deviation of FFI is an index which indicates a degree of pulse-wave-waveform interval dispersion. The smaller the standard deviation of FFT is, the more it becomes possible for the print device 1 to decide that the user tends to be nervous. In addition, the larger the standard deviation of FFT is, the more it becomes possible for the print device 1 to decide that the user tends to be relaxed.

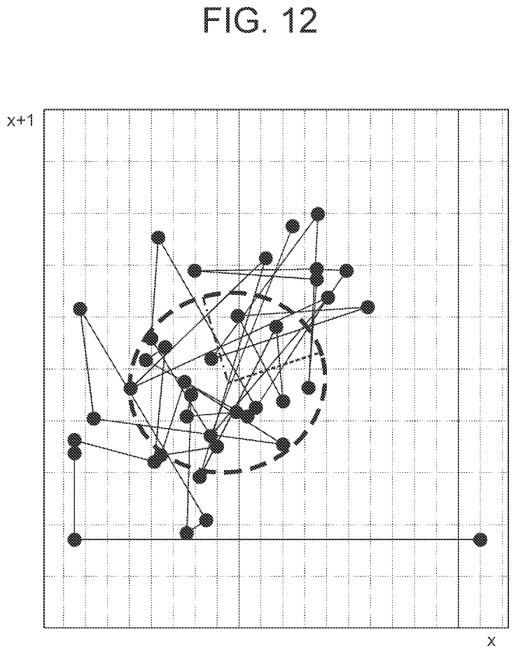

Next, the dispersion change rate of Lorenz plot will be described by using FIG. 12. The dispersion change rate of Lorenz plot is an index which indicates an autonomic nerve tendency. The dispersion change rate of Lorenz plot is calculated as follows. First, the Lorenz plot will be described. The Lorenz plot is the one that an interval between two continuously generated and mutually adjacent pulse waves is plotted on a graph with one being set as the horizontal axis and the other being set as the vertical axis and it is possible to observe the dispersion of the interval between the pulse waves.

For example, in a case of paying attention to a certain k-th pulse wave, each interval between the k-th pulse wave and a k+1-th pulse wave which comes after the k-th pulse wave is plotted in a state of making the k-th pulse wave and the k+1-th pulse wave correspond to the vertical axis and the horizontal axis of the graph respectively. One example that the interval is plotted in this way is illustrated in FIG. 12. The smaller the fluctuation of the interval between the continuously generated pulse waves is, the more points gather in the center. On the other hand, the larger the fluctuation of the interval between the continuously generated pulse waves is, the more the points are scattered to the surroundings.

Here, the dispersion of Lorenz plot will be described. First, the Lorenz plot is evaluated with a numerical value. In one embodiment, for example, the Lorenz plot is digitized by projecting the Lorenz plot onto orthogonal axes and thereby the dispersion of Lorenz plot is calculated. Specifically, in the graph of the Lorenz plot, an axis which passes the center of data distribution of each Lorenz plot and passes a point that k becomes the same as k+1 is defined as a y-x axis. In addition, an axis which passes the center of data distribution of each Lorenz plot and is orthogonal to the y-x axis is defined as a y-(−x) axis.

Each Lorenz plot of data which is obtained in one to three minutes is projected onto the above-described two axes respectively and one is defined as dispersion σ(x) on the y-x axis and the other is defined as dispersion σ(−x) on the y-(−x) axis. In this case, the dispersion of Lorenz plot is evaluated as an area of ellipse S=π×σ(x) x σ(−x). The larger the area of the ellipse is, the larger the dispersion is and the smaller the area of the ellipse is, the smaller the dispersion is.

The dispersion change rate of Lorenz plot is calculated by dividing the dispersion of Lorenz plot at the time of pulse wave measurement by the dispersion of Lorenz plot which is obtained in a usual state. In one embodiment, the storage unit 19 stores the dispersion of Lorenz plot which is obtained in the usual state in advance and the print device 1 calls the dispersion of Lorenz plot which is obtained in the usual state and that the storage unit 19 holds out of the storage unit 19 when calculating the dispersion change rate of the Lorenz plot and uses the called-out dispersion of Lorenz plot which is obtained in the usual state in calculation of the dispersion change rate of Lorenz plot.

In addition, the control unit 110 estimates the user's psychosomatic state by comparing the dispersion change rate of Lorenz plot with the threshold value. In a case where the calculated dispersion change rate of Lorenz plot exceeds the threshold value, it becomes possible for the print device 1 to decide that the user tends to relax. In addition, in a case where the calculated dispersion change rate of Lorenz plot is less than the threshold value, it becomes possible for the print device 1 to decide that the user tends to get nervous.

Next, the change rate of the pulse rate will be described. The change rate of the pulse rate is the index which indicates the tendency of the autonomic nerves. In addition, the pulse rate is the number of heart beats in a predetermined time. For example, the number of heart beats in one minute is defined as the pulse rate. The change rate of the pulse rate is calculated by dividing the pulse rate which is obtained in pulse wave measurement by the pulse rate which is obtained in the usual state. In one embodiment, the storage unit 19 stores the usual-state pulse rate in advance. In a case of calculating the change rate of the pulse rate, the print device 1 calls the usual-state pulse rate that the storage unit 19 holds out of the storage unit 19 and uses the called-out pulse rate in calculation.

In addition, the control unit 110 compares the change rate of the pulse rate with the threshold value and thereby estimates the user's psychosomatic state. For example, in a case where the calculated change rate of the pulse rate exceeds the threshold value, it becomes possible for the print device 1 to decide that the user tends to get nervous. In addition, in a case where the calculated change rate of the pulse rate is less than the threshold value, it becomes possible for the print device 1 to decide that the user tends to relax.

Next, the change rate of PA (Pulse Amplitude) is the index which indicates increase/decrease in amplitude of the pulse wave waveform. It is possible to calculate the change rate of PA as expressed in the following formula.

The change rate of PA=(PA2/PA1) (Formula)

PA1: a mean value of PA values which are measured in the usual state

PA2: a mean value of PA values which are measured in a predetermined time in measurement

The mean value of PA values which are measured in the usual state is measured in advance and is stored in the storage unit 19 of the print device 1. When calculating the biological information, the control unit 110 calls the mean value of the PA values which are measured in the usual state out of the storage unit 19 and performs calculation by using a numerical value of that mean value. In addition, the smaller the PA change rate is, the more it becomes possible for the print device 1 to decide that the user is in a poor blood circulation tendency. In addition, the larger the PA change rate is, the more it becomes possible for the print device 1 to decide that the user is in a good blood circulation tendency.

Next, the baseline change rate is the index which relates to the blood circulation state. It is possible to calculate the baseline change rate as expressed in the following formula.

Baseline change rate=(BL2/BL1) (Formula)

BL1: baseline of pulse wave information which is obtained in the usual state

BL2: baseline of pulse wave information which is obtained by measurement

Here, the baseline is a mean value of reduced luminance values in a predetermined time. In addition, in one embodiment, the baseline of the pulse wave information which is obtained in the usual state is measured in advance and is stored in the storage unit 19 of the print device 1. In calculation of the biological information, the control unit 110 calls the usual-state baseline out of the storage unit 19 and performs calculations by using the numerical value of the usual-state baseline. In a case where the change rate of the baseline is less than the threshold value, it is possible for the print device 1 to decide that the blood circulation of the user is poor. In addition, in a case where the change rate of the baseline exceeds the threshold value, it is possible for the print device 1 to decide that the blood circulation of the user is good.

Next, respective meanings of the baseline and PA will be considered. As described above, the principle of extraction of the pulse wave information from the luminance of the video lies in the point of capturing a temporal change in the luminance of the green light which is absorbed by the hemoglobin. Accordingly, it is thought that the baseline is in an almost proportional relation with an average of amounts of the hemoglobin in a target part which is obtained in a measurement period. That is, it is possible to interpolate a change in baseline as a change in an average blood volume in that target part to be measured. In contrast, a pulse wave amplitude itself indicates pulsation of pulses and therefore it is possible to interpret a change in PA as a change in strength of the pulsation.

Incidentally, the control unit 110 pertaining to one embodiment makes the storage unit 19 store data which is obtained in the usual state as a standard for calculation when calculating the dispersion change rate of Lorenz plot, the change rate of the pulse rate, the change rate of PA and the change rate of the baseline. However, the data which is used as the standard of calculation is not limited to the data which is obtained in the usual state and, for example, a mean value of other users who belong to the generation which is the same as the generation of the user concerned may be used and a mean value of other users who are the same as the user concerned in gender may be used. In addition, the video processing section 111 may acquire data two or more times in execution of print processing and may use one piece of the data which is acquired as a standard of calculation.

Next, CVRR (Coefficient of Variation of R-R intervals) is the index which indicates a fluctuation of interval between pulse wave pulses and indicates the state of the heart rate adjustment function. In addition, CVRR is calculated by frequency analysis and so forth of the pulse wave information. The smaller CVRR is, the more it becomes possible for the print device 1 to decide that the user's heart rate adjustment function is in an inferior state. In addition, the larger CVRR is, the more it becomes possible for the print device 1 to decide that the heart rate adjustment function of the user is in a favorable state.

Next, the strain time is the index which indicates the relative blood pressure fluctuation. In addition, in the pulse wave information, the strain time is a time lag between a pulse wave component and a waveform strain component which is induced by the blood pressure fluctuation. The shorter the strain time is, the more it becomes possible for the print device 1 to decide that the user's blood pressure is in a high state. In addition, the longer the strain time is, the more it becomes possible for the print device 1 to decide that the user's blood pressure is in a low state. Incidentally, it is possible for the print device 1 pertaining to one embodiment to calculate various indexes which indicate the user's psychosomatic state on the basis of the acquired pulse wave information, not limited to the above indexes.

### [Printing Operation]

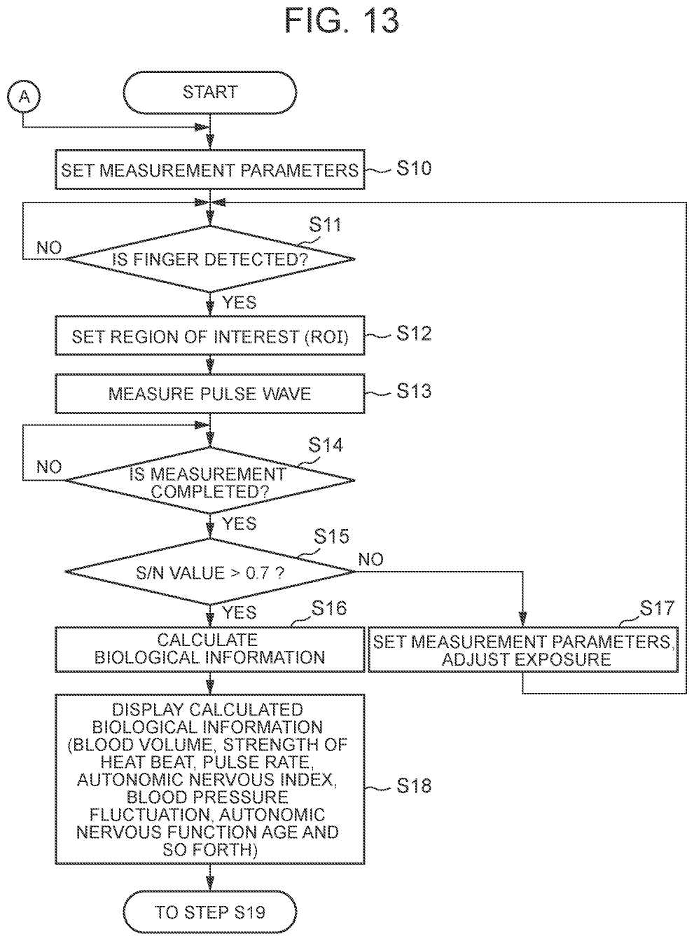

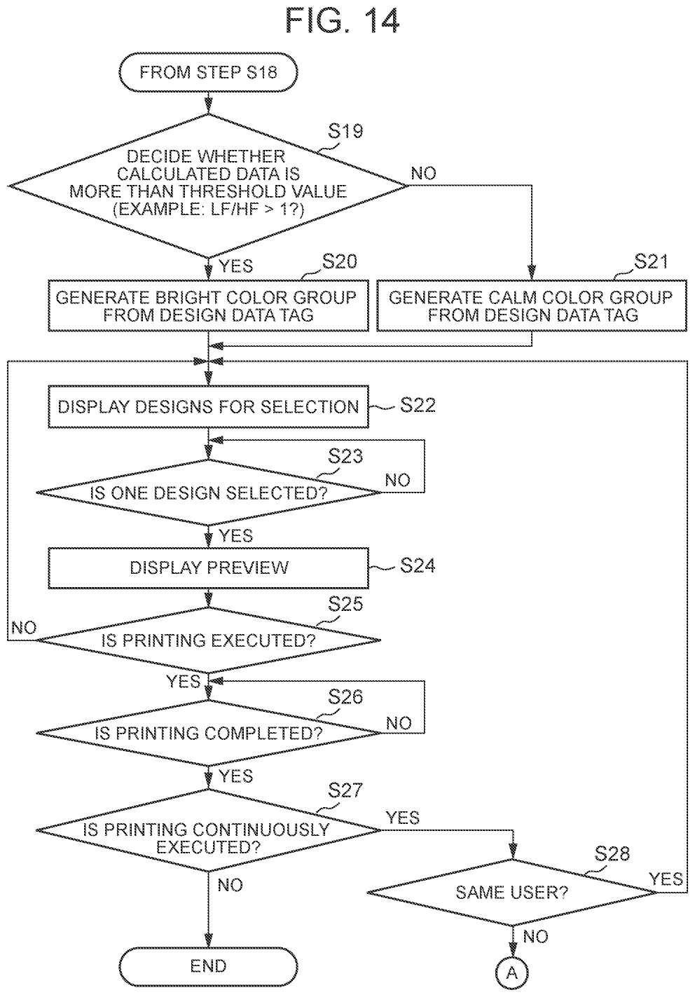

Next, a flow of print processing will be described with reference to FIG. 13 and FIG. 14. FIG. 13 and FIG. 14 illustrate one example of a flowchart for explaining a flow of processing that the print device 1 which has the functional configuration in FIG. 4 and is illustrated in FIG. 2 executes. Execution of the print processing is started by an operation of starting the print processing that the user performs on the input unit 56 of the user terminal 2 in a state where the user's finger 5 is placed on the finger placement stand 32b.

First, as illustrated in FIG. 13, the control unit 110 sets measurement parameters of the image pickup unit 16 to initial values which are set in advance (step S10). The measurement parameters are set values which relate to image-picking-up by the camera 16a of the image pickup unit 16. The set values are numerical values which relate to settings of, for example, exposure, a focal distance and magnification of the camera 16a, and brightness, luminance and so forth of the LED of the lamp 16b. It becomes possible to reduce the noise in the video and to increase reliability of the pulse wave information to be measured by appropriately setting the measurement parameters.

In addition, there are cases where the luminance of the video varies under the influence of the skin color and so forth of the user whose video is to be picked up. Therefore, in a case of assuming individual differences among users who use the print devices 1, the respective measurement parameters may not be initial values and, instead, the measurement parameters may be set in accordance with each user. For example, in step S11, a video of the finger 5 on the finger placement stand 32b is picked up and then the set values may be changed on the basis of the picked-up video of the finger 5. Further, the control unit 110 may save the measurement parameters in units of individuals into the authentication server in the server group 4 over the network 3 at a timing of completion of execution of the print processing and may call the measurement parameters out of the authentication server and may set the measurement parameters when starting execution of another print processing.

In one embodiment, the control unit 110 performs setting of the measurement parameters every time execution of the print processing is started. However, in a case where the control unit 110 makes the storage unit 19 hold the measurement parameters which are set and executes the print processing on the same user in the next and subsequent times, the control unit 1110 may use the measurement parameters which are held in the storage unit 19.

Next, the control unit 110 decides whether the user's finger 5 is detected by the sensor of the print unit 32 (step S11). In a case where the user's finger 5 is detected by the sensor (step S11: Yes), the control unit 110 sets the region of interest ROI on the user's finger 5 (step S12).

Next, the control unit 110 makes the image pickup unit 16 start picking-up of the video of the finger 5 and thereby starts measurement of the user's pulse wave (step S13). At completion of measurement of the user's pulse wave (step S14: Yes), the control unit 110 calculates an S/N (Signal to Noise) value of the measured pulse wave information and compares the calculated S/N value with the threshold value (step S15).

Here, the S/N value is the index which is used for confirmation of how much noise other than pulse waves is contained in the measured pulse wave information. The smaller the S/N value is, the more it is possible to decide that acquired video data is reliable. In one embodiment, the threshold value is set to, for example, 0.7 thereby to define data of the S/N value which is less than 0.7 as reliable data. Incidentally, the threshold value is not limited to 0.7 and it is possible to set any value as the threshold value in accordance with an object of a device to be applied.

In a case where the S/N value is less than 0.7 (step S15: No), the control unit 110 makes the image pickup unit 16 adjust again the measurement parameters such as the exposure and so forth of the camera 16a (step S17) and then shifts the processing to step S11. On the other hand, in a case where the S/N value exceeds 0.7 (step S15: Yes), the control unit 110 calculates data to be used for decision in the biological information which is indicated in FIG. 10 (step S16). Then, the control unit 110 transmits the calculated data to be used for decision in the biological information to the user terminal 2 via the communication unit 20 in order to display the calculated data to be used for decision in the biological information on the user terminal 2 (step S18).

Next, the control unit 110 makes decision by comparing the calculated data to be used for decision in the biological information with the threshold values respectively as illustrated in FIG. 14 (step S19). In one embodiment, setting is made so as to use LF/HF as the index for decision, to decide nail designs which express the user's psychosomatic state and to present the decided nail designs to the user. For example, the control unit 110 decides whether a numerical value of LF/HF is less/more than a threshold value which is set to, for example, “1”. In a case where the LF/HF value is more than the threshold value “1” (step S19: Yes), the control unit 110 prepares a group of bright-color nail designs from tags in the design data as decision information to be used for nail design selection and transmits the decision information to the user terminal 2 via the communication unit 20 (step S20).

Then, the control unit 70 of the user terminal 2 issues a command signal to the nail design registration/distribution server in the server group 4 via the communication unit 59 and makes the nail design registration/distribution server transmit data on the nail designs which are tagged with the bright colors to the user terminal 2. The control unit 70 of the user terminal 2 prepares a plurality of groups of nail designs which are decided by using the received nail design data. The control unit 70 prepares data which contains, for example, two groups of the nail designs such as the groups which are illustrated in FIG. 11. Incidentally, there is no specific limitation on the number of the nail designs to be prepared.

Further, the control unit 110 transmits a command signal for display of a design selection screen to the user terminal 2 via the communication unit 20 in order to display the design selection screen to be used for nail design selection which is made by the user (step 22).

The input processing section 73 of the user terminal 2 accepts nail design selection that the user performs by using the input unit 56. The control unit 70 of the user terminal 2 transmits information on nail design selection to the print device 1 via the communication unit 59.

The control unit 110 confirms whether the communication unit 20 (the communication processing section 71) receives the nail design selection information (step S23). In a case where the communication processing section 71 receives the nail design selection information (step S23: Yes), the control unit 110 shifts the processing to step S24.

On the other hand, in a case where the LF/HF value is less than the threshold value “1” (step S19: No), the control unit 110 outputs information that the LF/HF value is less than “1” to the communication processing section 116 and thereby transmits decision information which is used to generate a group of calm-color nail designs to be used for design selection from the tags of design data to the user terminal 2 via the communication unit 20 (step S21).

Then, the control unit 70 of the user terminal 2 issues a command signal to the nail design registration/distribution server in the server group 4 via the communication unit 59 and makes the nail design registration/distribution server transmit data on nail designs which are tagged with the calm colors to the user terminal 2. The control unit 70 of the user terminal 2 prepares a plurality of groups of nail designs which is determined by using the received nail design data.

Further, the control unit 110 transmits a command signal for display of a design selection screen to the user terminal 2 via the communication unit 20 in order to display the design selection screen which is used for nail design selection on a screen of the output unit 57 of the user terminal 2 (step 22).

The input processing section 73 of the user terminal 2 accepts nail design selection information which is input into the input unit 56 by the user. The control unit 70 of the user terminal 2 transmits the nail design selection information to the print device 1 via the communication unit 59.

The control unit 110 confirms whether the communication unit 20 receives the nail design selection information (step S23). In a case where the communication processing section 71 receives the nail design selection information (step S23: Yes), the control unit 110 shifts the processing to step S24.

Next, the control unit 110 prepares a preview video that the selected nail design is superimposed on the picked-up video of the finger 5. In addition, the control unit 110 operates so as to superimpose also a selection screen for asking whether nail printing is to be executed on the preview video. In addition, the control unit 110 transmits the prepared preview video to the user terminal 2 via the communication unit 20 so as to display the preview screen on the user terminal 2 (step S24).

The control unit 70 of the user terminal 2 makes the output unit 57 display the received preview video on its own screen. In addition, a selection screen for asking whether execution of the printing is selected is displayed on the screen of the output unit 57 of the user terminal 2. The user touches an option which is displayed on the screen and thereby the control unit 70 of the user terminal 2 accepts information for selecting whether the printing is executed. The control unit 70 of the user terminal 2 transmits the selection information to the print device 1 via the communication unit 59.

Next, the control unit 110 confirms whether the printing is to be executed from the received the selection information (step S25). In a case where the selection information indicates selection of no execution of the printing (step S25: No), the control unit 110 shifts the processing to step S22. In a case where the selection information indicates selection of execution of the printing (step 25: Yes), the control unit 110 makes the print unit 32 print the selected nail design on the nail 5a of the user's finger 5. Then, the control unit 110 confirms whether the printing operation which is executed by the print unit 32 is completed (step S26).

In a case where the printing operation which is executed by the print unit 32 is completed (step S26: Yes), the control unit 110 transmits a command signal for encouraging display of a screen for selecting whether the printing is continuously executed to the user terminal 2 via the communication unit 20.

The control unit 70 of the user terminal 2 makes the output unit 57 display the screen for selecting whether the printing is continuously executed on its own screen. The user touches an option which is displayed on the screen and thereby the control unit 70 of the user terminal 2 accepts the information for selecting whether the printing is continuously executed. The control unit 70 of the user terminal 2 transmits that selection information to the print device 1 via the communication unit 59. Incidentally, in continuous execution of the printing, for example, printing on a nail 5a of another finger 5 of the same user and using of the print device 1 by another user are assumed.

The control unit 110 confirms the received information for selecting whether the printing is continuously executed (step S27). In a case where the selection information indicates selection of continuous execution of the printing (step S27: Yes), the control unit 110 transmits a command signal for encouraging display of a selection screen for confirming whether a user who continuously executes the printing is the same user to the user terminal 2 via the communication unit 20.

The control unit 70 of the user terminal 2 makes the output unit 57 display a screen for selecting whether the printing is continuously executed on its own screen. The user touches an option which is displayed on the screen and thereby the control unit 70 of the user terminal 2 accepts the information for selecting whether the printing is continuously executed. The control unit 70 of the user terminal 2 controls to transmit the information for selecting to the print device 1 via the communication unit 59.

The control unit 110 confirms the selection information which is transmitted from the user terminal 2 (step S28). In a case where the selection information indicates that the user who continuously executes the printing is the same user (step S28: Yes), the control unit 110 shifts the processing to step S22. Thereby, it becomes possible for the user to print the nail design on the nail 5a of another finger 5 by omitting execution of processing such as measurement parameter setting and so forth.

On the other hand, in a case where the selection information indicates that the user who continuously executes the printing is not the same person (step S28: No), the control unit 110 shifts the processing to step S10. Thereby, it becomes possible for the print unit 110 to execute the print processing on the nail 5a of a new user, starting from setting of the measurement parameters. Next, a case where “No” is set in step S27 will be described. In a case where information which indicates selection of termination of the printing without continuous execution of the printing is input, (step S27: No), the control unit 110 terminates execution of the print processing. Incidentally, in one embodiment, the control unit 110 of the print device 1 executes processes in step S19 to step S28 which are indicated in FIG. 14. As an alternative, the control unit 70 of the user terminal 2 may execute these processes.

The print device 1 which is configured as described above includes the control unit 110 which acquires the biological information which relates to the blood flow and determines the nail designs to be presented to the user on the basis of the acquired biological information.

Thereby, it becomes possible for the print device 1 pertaining to one embodiment to present the nail designs which are more suited for the user's psychosomatic state to the user.

In addition, the control unit 110 acquires the biological information on the basis of the information which relates to the video which is obtained by picking up an image of at least part of the user's body. In addition, the biological information is video-based photoplethysmography.

Thereby, it becomes possible for the print device 1 pertaining to one embodiment to acquire the biological information with no contact with the user.

In addition, the print device 1 acquires the biological information on the basis of the information on the video which is obtained by picking up the image of the user's finger.

Thereby, it becomes possible for the print device 1 pertaining to one embodiment to perform the pulse wave measurement by using the video which is taken by a camera that a nail printer includes and to lead this pulse wave measurement to weight saving and cost reduction which are attained by a reduction in the number of components of a nail printer. In addition, when printing the nail designs by the nail printer, it becomes possible for the print device 1 to perform the pulse wave measurement simultaneously with printing of the nail designs by the nail printer. Thereby, it becomes possible for the print device 1 to save time and labor which are taken for the pulse wave measurement. Further, in the nail printer, a location that the finger is placed is a place where external light and so forth which induce noise generation in the video pulse wave are not incident. Accordingly, it becomes possible for the print device 1 to reduce noises in the video which is used for pulse wave measurement.

In addition, the control unit 110 decides the user's blood circulation state on the basis of the biological information and determines the nail designs to be presented to the user on the basis of the decided blood circulation state.

Thereby, it becomes possible for the print device 1 pertaining to one embodiment to provide the nail designs which are appropriate for the blood circulation state of the user. For example, it becomes possible for the print device 1 to provide the nail designs which express the blood circulation state of the user. It becomes also possible for the print device 1 to present calm-color nail designs to the user in a case where the user's blood circulation is poor and to present blight-color nail designs to the user in a case where the user's blood circulation is good and thereby to make the user recognize his/her psychosomatic state.

In addition, the control unit 110 decides the nervous state of the user on the basis of the biological information and determines the nail designs to be presented to the user on the basis of the decided nervous state of the user.

Thereby, it becomes possible for the print device 1 pertaining to one embodiment to provide the nail designs which are appropriate for the nervous state of the user. For example, it is possible for the print device 1 to provide nail designs which are effective for suppressing depressing feeling of the user and keeping mental balance. It is also possible for the print device 1 to color each nail design with a color which has a calming-down effect in a case where the user is in the nervous state and to color each nail design with a color which has a mind-lifting-up effect in a case where the user is in a relaxed state and thereby to contribute to achievement of the mental balance of the user.

In addition, the control unit 110 decides the user's heart rate adjustment function state on the basis of the biological information and determines the nail designs to be presented to the user on the basis of the decided heart rate adjustment function.

Thereby, it becomes possible for the print device 1 pertaining to one embodiment to provide the nail designs which are appropriate for the user's heart rate adjustment function state. For example, it becomes possible for the print device 1 to provide nail designs which express the user's heart rate adjustment function state. It becomes possible for the print device 1 to color the nail designs with calm colors in a case where the user's heart rate adjustment function state is poor and to color the nail designs with bright colors in a case where the user's heart rate adjustment function state is good and thereby to make the user recognize his/her psychosomatic state.

In addition, the control unit 110 decides a high/low state of the user's blood pressure on the basis of the biological information and determines the nail designs to be presented to the user on the basis of the decided high/low state of the user's blood pressure.

Thereby, it becomes possible for the print device 1 pertaining to one embodiment to provide the nail designs which are appropriate for the user's blood pressure state. For example, it becomes possible for the print device 1 to provide the nail designs which express the user's blood pressure state. For example, it is possible for the print device 1 to color the nail designs with the calm colors in a case where the user's blood pressure is low and to color the nail designs with the bright colors in a case where the user's blood pressure is high and thereby to make the user recognize his/her psychosomatic state.

In addition, in the print device 1 pertaining to one embodiment, the groups each of which is configured by the plurality of nail designs are set in advance and the control unit 110 determines the nail designs to be presented to the user in units of groups on the basis of the biological information.

Thereby, it becomes possible for the print device 1 to more reduce the time which is taken for determination of the nail designs to be presented to the user.

### MODIFIED EXAMPLES

Incidentally, the present invention is not limited to the above-described embodiment and modification, improvement and so forth which are made within a range that it is possible to attain the object of the present invention fall within the range of the present invention. For example, it is possible to modify the above-mentioned embodiment and thereby to present it as modified examples such as those which are exemplified in the following. In addition, it is also possible to combine the modified examples such as those which are exemplified in the following with one another.

### First Modified Example

In the above-mentioned embodiment, the print device 1 calculates the biological information such as LF/HF, the standard deviation of FFI, the dispersion range rate of the Lorentz plot, the change rate of the pulse number, the change rate of PA, the change rate of the baseline, CVRR, the strain time and so forth. However, biological information to be calculated is not limited to the above biological information. For example, in a configuration to which a cuff which presses the finger is added, it is possible to calculate a blood volume from pulse wave information which is obtained before and after the finger is pressed by measuring the pulse waves in both cases where the finger is pressed with the cuff and the finger is not pressed with the cuff. Thereby, it becomes possible for the print device 1 to present the nail designs which are appropriate for the blood volume to the user.

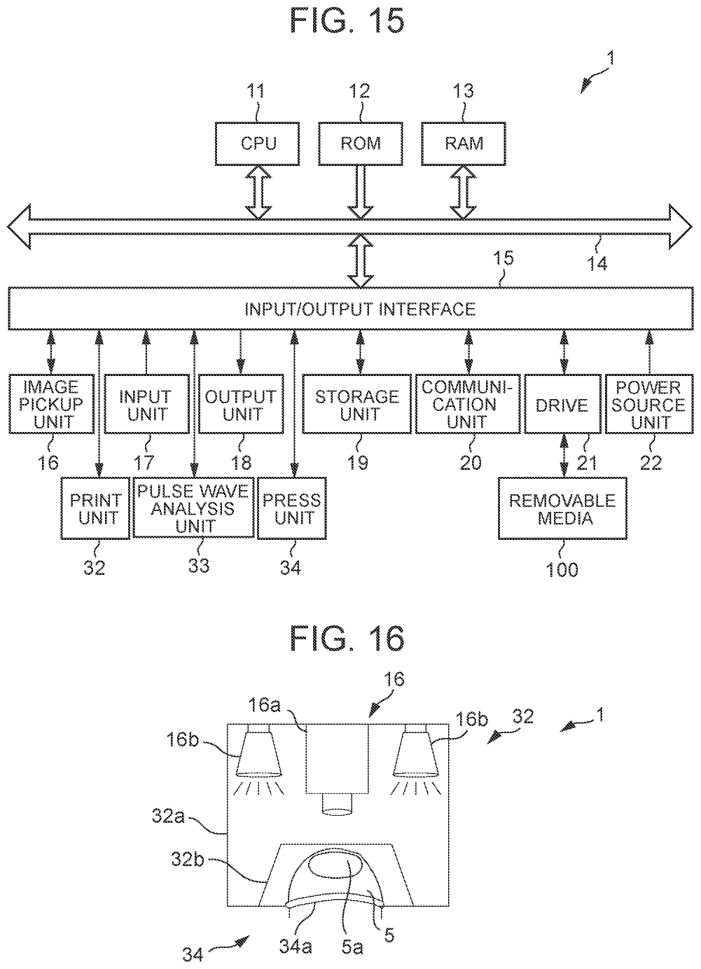

A hardware configuration of the print device 1 pertaining to the first modified example will be described by using FIG. 15. Incidentally, there are cases where the same term is assigned to a configuration which is common to or the same as an already described configuration and detailed description thereof is omitted. FIG. 15 is a block diagram illustrating the hardware configuration of the print device 1 pertaining to the first modified example.

The print device 1 pertaining to the first modified example is a computer which includes the CPU 111, the ROM 12, the RAM 13, the bus 14, the input/output interface 15, the image pickup unit 16, the input unit 17, the output unit 18, the storage unit 19, the communication unit 20, the drive 21, the power supply unit 22, the print unit 32, the pulse wave analysis unit 33 and a press unit 34. Incidentally, as described above, there are cases where the same term is assigned to the configuration which is common to or the same as the configuration which has already been described and detailed description thereof is omitted.

The press unit 34 has a cuff 34a which is in the form of an elongated cloth bag which is illustrated in FIG. 16 and a not-illustrated pump section which pumps air into the cuff 34a. FIG. 16 illustrates one example of a state where the cuff 34a is wound around the finger 5 which is placed on the finger placement stand 32b in the print unit 32 of the print device 1. It is possible for the press unit 34 to adjust the blood flow by making the pump section pump the air into the cuff 34a so as to inflate the cuff 34a with the air and thereby pressing the blood vessels of the finger 5 in a state where the cuff 34a is wound around the finger 5 of the user. Incidentally, although the finger is pressed by pumping the air into the cuff 34a in the first modified example, the present invention is not limited to this way of pressing the finger. For example, the user's finger 5 may be pressed by binding the user's finger 5 with a tube and so forth.

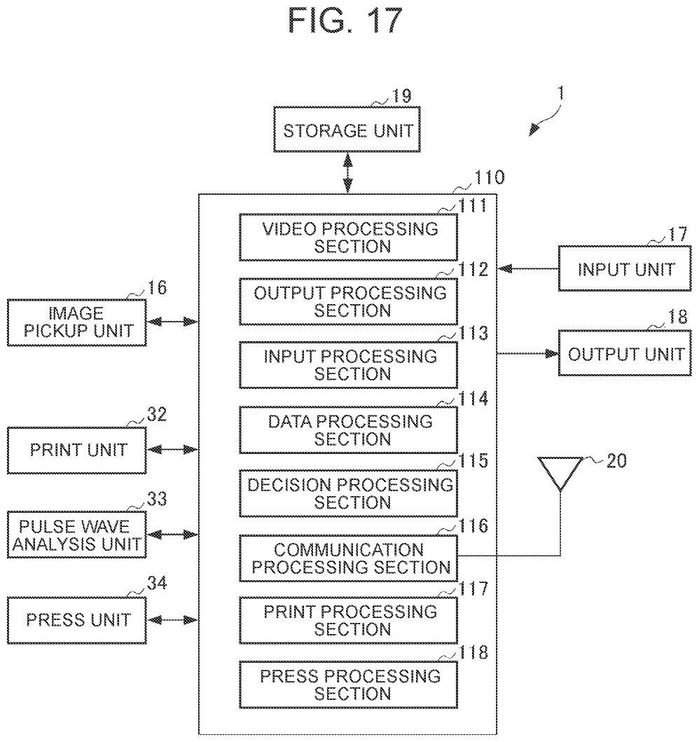

Next, a functional configuration of the print device 1 pertaining to the first modified example will be described. FIG. 17 is a functional block diagram illustrating one example of part of the functional configuration of the print device 1. The control unit 110 which executes various control processing is realized by the CPU 11 which executes arithmetic processing. The control unit 110 has the video processing section (the video processing function) 111, the output processing section (the output processing function) 112, the input processing section (the input processing function) 113, the data processing section (the data processing function) 114, the decision processing section (the decision processing function) 115, the communication processing section (the communication processing function) 116, the print processing section (the print processing function) 117 and a press processing section (a press processing function) 118. Incidentally, there are cases where the same term is assigned to the configuration which is common to or the same as the already-described configuration and detailed description thereof is omitted.

It is possible for the press processing section 118 to execute control processing for driving the pump section of the press unit 34 and pumping the air into the cuff 34a. For example, in the first modified example, in a case where the pulse wave measurement is performed in a state where the user's finger 5 is being pressed with the cuff 34a, the press processing section 118 receives a command to start pressing of the finger 5 with the cuff 34a and drives the pump section of the press unit 34.

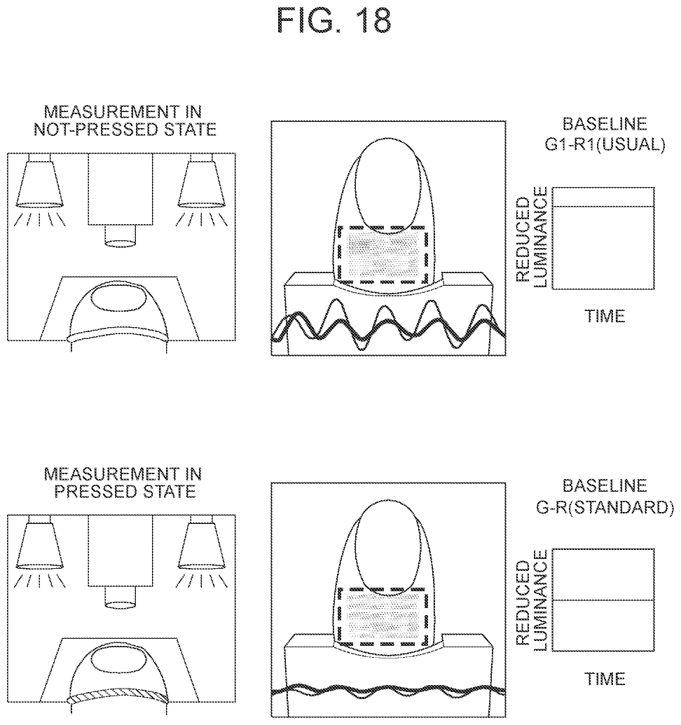

FIG. 18 is explanatory diagrams illustrating examples of a difference between respective pulse wave measurement results which are obtained depending on whether the finger 5 is pressed with the cuff 34a. Upper-stage diagrams illustrate examples of the pulse wave measurement result which is obtained in a state where the finger 5 is not pressed with the cuff 34a and lower-stage diagrams illustrate examples of the pulse wave measurement result which is obtained in a state where the finger 5 is pressed with the cuff 34a.

Specifically, the upper-stage left diagram illustrates one example of a measurement state in a case where the pulse wave of the finger 5 is measured in a state where the finger 5 is not pressed with the cuff 34a. The upper-stage center diagram illustrates one example of a state where the region of interest ROI and a pulse wave waveform are superimposed on a video of the finger 5 which is obtained while the pulse wave is being measured in the state where the finger 5 is not pressed with the cuff 34a. The upper-stage right diagram is one example of a graph which indicates the baseline in a case where the pulse wave measurement is performed in the state where the finger 5 is not pressed with the cuff 34a. In the first modified example, the baseline which is measured in the state where the finger is not pressed with the cuff 34a is set as a usual baseline, a calculation is performed by subtracting a red signal R1 from a green signal G1 and thereby G1-R1 is obtained.

In addition, specifically, the lower-stage left diagram illustrates one example of a measurement state in a case where the cuff 34a is inflated and the finger 5 is pressed with the cuff 34a and then the pulse wave measurement of the finger 5 is performed. The hatched cuff 34a indicates that the cuff 34a is in a finger-pressing state. The lower-stage central diagram illustrates one example of a state where the region of interest ROI and the pulse wave waveform are superimposed on the video of the finger 5 the pulse wave of which is being measured in the pressed state. The lower-stage right diagram illustrates one example of a graph which indicates the baseline in a case where the pulse wave measurement is performed in the state where the finger 5 is pressed with the cuff 34a. In the first modified example, the baseline which is measured in the state where the finger 5 is pressed with the cuff 34a is set as a standard baseline and a calculation is performed by subtracting the red signal R from the green signal G and thereby G-R is obtained.

As illustrated in the upper-stage and lower-stage left diagrams in FIG. 18, the pulse wave measurement which is performed in the state where the finger 5 is pressed with the cuff 34a is performed under the condition which is the same as the condition in the upper-stage left diagram, excepting that the finger 5 is pressed with the cuff 34a. As is apparent from a comparison between the upper-stage and lower-stage central diagrams, it is found that a pulse-wave amplitude of the video in the lower-stage central diagram is smaller than a pulse-wave amplitude of the video in the upper-stage central diagram. That is, it is found that the pulse-wave amplitude becomes small because the finger 5 is pressed with the cuff 34a. As illustrated in the upper-stage and lower-stage left diagrams in FIG. 18, when comparing the calculated baselines with each other, the baseline G-R which is based on the pulse wave information which is obtained in the state where the finger 5 is pressed with the cuff 34a is smaller than the baseline G1-R1.

In the first modified example, it is possible for the control unit 110 to calculate the blood volume which is increased when measuring the pulse wave in comparison with the blood volume which is obtained in a standard state by subtracting the baseline (G-R) which is obtained from the pulse wave measurement which is performed in the finger-pressed state from the usual baseline (G1-R1) which is obtained from the pulse wave measurement which is performed with no press on the finger 5.

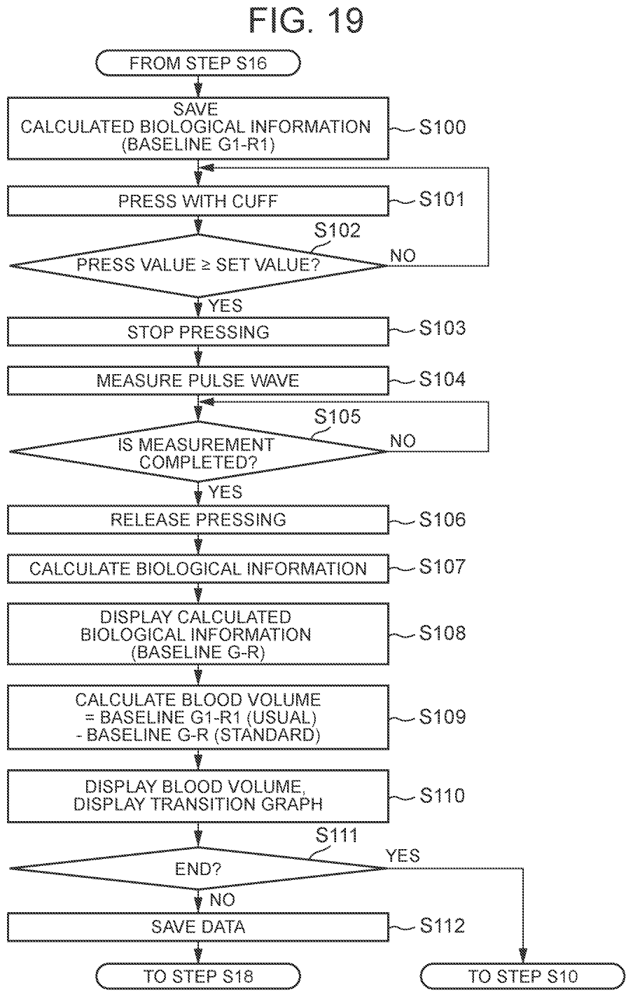

Next, concrete processing which is executed in the first modified example will be described. FIG. 19 is part of a flowchart explaining one example of a flow of print processing which is executed by the print device 1. Step S10 to step S16 and step S18 to step S28 are common to one embodiment and the first modified example of the present invention and therefore a flowchart and detailed description of these steps are omitted.

Description will be made, starting from step S16 in FIG. 13. The control unit 110 calculates the various kinds of biological information from the acquired pulse wave information (step S16). Next, the control unit 110 saves the generated biological information, for example, the calculated baseline (G1-R1) (step S100). Next, the control unit 110 makes the press unit 34 drive the pump section and makes the press unit 34 start pressing of the finger 5 by pumping air into the cuff 34a which is wound around the finger 5 (step S101).

The control unit 110 acquires a pressing value (a pressure) in the cuff 34a from the press unit 34 and confirms whether the pressing value exceeds a set value which is set in advance (step S102). In a case where the pressing value exceeds the set value (step S102: Yes), the control unit 110 makes the press unit 34 stop the operation of the pump section and thereby makes the press unit 34 stop pressing of the finger 5 (step S103). Then, the control unit 110 makes the image pickup unit 16 start picking-up of the image of the finger 5 and starts the pulse wave measurement (step S104).

At completion of the pulse wave measurement (step S105: Yes), the control unit 110 makes the press unit 34 release the air in the pump section and thereby releases pressing of the finger 5 (step S106). The control unit 110 operates to calculate the various kinds of biological information from the pulse wave which is measured in step S104 and step S105 (step S107). Next, the control unit 110 transmits the biological information and a command signal to the user terminal 2 via the communication unit 20 in order to display the calculated biological information on the screen of the output unit 57 of the user terminal 2 (step S108).

The control unit 70 of the user terminal 2 displays the calculated biological information such as, for example, the information (G-R) on the calculated baseline on the screen of the output unit 57 on the basis of the acquired biological information and command signal.

Then, the control unit 110 calculates the blood volume by subtracting the calculated baseline information (G-R) from the saved baseline information (G1-R1) (step S109). Next, the control unit 110 transmits the calculated various kinds of biological information and a command signal to the user terminal 2 via the communication unit 20 in order to display the calculated blood volume and a transition graph on the screen of the output unit 57 of the user terminal 2.

The control unit 70 of the user terminal 2 makes the output unit 57 display the calculated blood volume and the transition graph on its own screen on the basis of the various kinds of biological information and the command signal which are acquired (step S110). Next, the control unit 110 transmits a command signal for encouraging display of a screen for selecting whether a print job for printing the nail design on the nail 5a is terminated to the user terminal 2.

The control unit 70 of the user terminal 2 receives the command signal and then makes the output unit 57 display the screen for selecting whether the print job is terminated on its own screen. The user touches an option which is displayed on the screen of the output unit 57 and thereby the control unit 70 of the user terminal 2 accepts user's selection information. In addition, the control unit 70 of the user terminal 2 transmits selection information as to whether the print job is to be terminated or continued to the print device 1 via the communication unit 59.

Next, the control unit 110 confirms the selection information which is received via the communication unit 20 (step S111). In a case where the selection information indicates selection of no-termination of the print job (step S111: No), the control unit 110 makes the storage unit 19 store the calculated biological information and shifts the processing to step S18 (step S112). In a case where the selection information indicates selection of termination of the print job (step S111: Yes), the control unit 110 shifts the processing to step S10. After that, the processing which is the same as the processing which is described with reference to FIG. 13 in one embodiment is executed.

As described above, the print device 1 pertaining to the first modified example includes the constitutional element which presses the finger 5 in addition to the constitutional elements in one embodiment of the present invention. The print device 1 operates to measure the pulse waves which are obtained in a case where the finger 5 is pressed with the cuff 34a and in a case where the finger 5 is not pressed with the cuff 34a and to calculate the blood volume. Thereby, it becomes possible for the print device 1 to present the nail designs which are appropriate for the blood volume which indicates the user's psychosomatic state.

### Second Modified Example

In the first modified example, in controlling to stop pressing of the finger 5 by the press unit 34 in the print processing (step S102 in FIG. 19), the set value which is set in advance is defined as the threshold value and whether finger pressing is to be stopped is decided by comparing the press value with the set value. However, the way of stopping finger-pressing is not limited to the above. The print device 1 also controls to stop pressing of the finger 5 also, for example, in a case where a level of noise in the pulse wave information exceeds a constant level. As a method of deciding the level of noise which is contained in the pulse wave information, a method of deciding the noise level by using the S/N value is available. Incidentally, since the print device 1 pertaining to the second modified example and the print device 1 pertaining to the first modified example are the same as each other in configuration elements and therefore detailed description of the print device 1 pertaining to the second modified example will be omitted by assigning the same terms to the same constitutional elements.

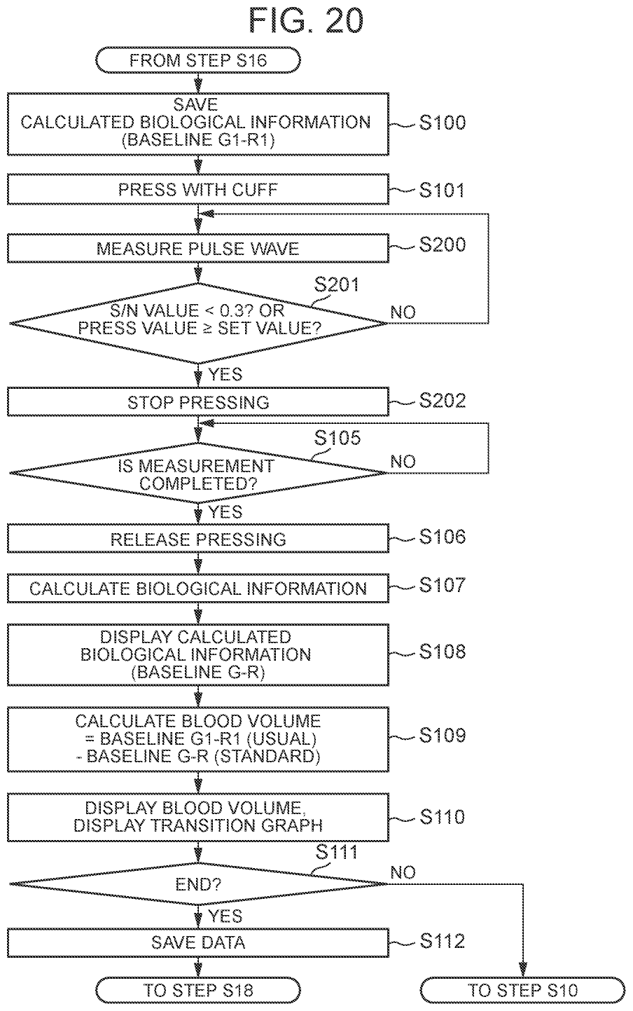

Concrete processing in the second modified example will be described. FIG. 20 is part of a flowchart for explaining one example of a flow of print processing which is executed by the print device 1. The flowchart in the second modified example is applied to processing that step S102 to step S104 in the first modified example are replaced with step S200 to step S202. Individual processes in other steps are common to the first and second modified examples and therefore a flowchart for these steps and detailed description thereof will be omitted.

First, step S101 will be described first. The control unit 110 makes the press unit 34 drive the pump section and start pressing of the finger 5 by pumping the air into the cuff 34a which is wound around the finger 5 (step S101). Next, the control unit 110 makes the image pickup unit 16 start picking-up of the image of the finger 5 and starts measurement of the user's pulse wave (step S200). Next, the control unit 110 confirms the S/N value of the acquired pulse wave and the press value (the pressure) of the cuff 34a (step S201). In a case where the S/N value is less than, for example, 0.3 or in a case where the press value exceeds the set value (step S201: Yes), the control unit 110 makes the press unit stop the operation of the pump section and stop pressing of the finger 5 (step S202). After that, the processing which is the same as the processing in the first modified example which is described with reference to FIG. 19 is executed. Incidentally, the threshold value of the S/N value is not limited to 0.3 and is freely settable depending on individual users.

As described above, in the print device 1 pertaining to the second modified example, when measuring the pulse waves in a case where the finger 5 is pressed and in a case where the finger 5 is no pressed and then calculating the blood volume, whether finger pressing is to be stopped is decided by watching an amount of noise in the pulse wave information. Thereby, it becomes possible for the print device 1 to perform the pulse wave measurement more accurately and to present the nail designs which are appropriate for the psychosomatic state of the user more precisely.

### Third Modified Example

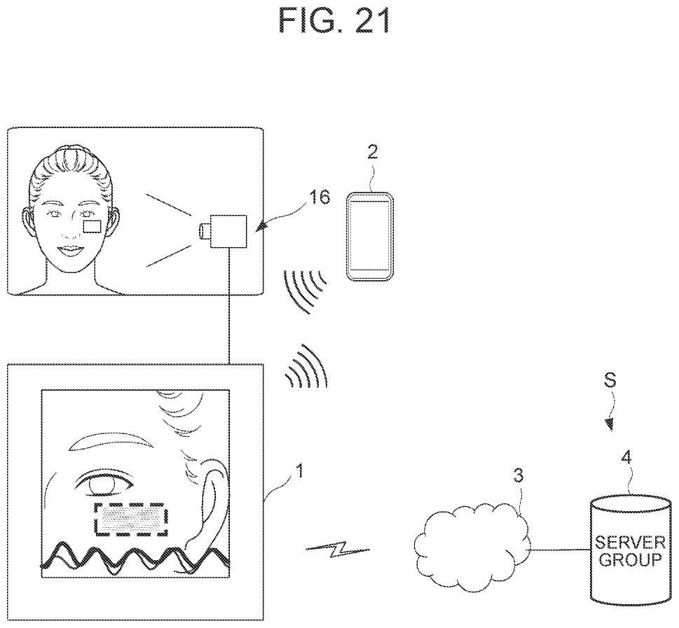

In one embodiment, the pulse wave information is acquired by analyzing the finger's video. However, the way of acquiring the pulse wave information is not limited to the above. For example, in the print device 1, the pulse wave information may be acquired by picking up a video of a user's face by a camera which is externally installed separately from the print device 1 as illustrated in FIG. 21 and analyzing the video of the user's face. Incidentally, the print device 1 pertaining to the third modified example is the same as the print device 1 pertaining to one embodiment except a point that the image pickup unit 16 is installed outside a housing of the print unit 32. Accordingly, the same terms are assigned to other elements which are the same as the elements in one embodiment and detailed description thereof is omitted. In addition, since the concrete processing is common to the third modified example and one embodiment, a flowchart and the detailed description of the processing are omitted.

As described above, in the print device 1 pertaining to the third modified example, the pulse wave information is acquired from the user's face video and the biological information is calculated on the basis of the acquired pulse wave information. Thereby, a freedom degree of design of the print device 1 is increased. In addition, although in the third modified example, the print device 1 picks up the user's face video, part to be picked up is not limited to the user's face. For example, the part to be picked up may be either a user's leg or a user's arm. In addition, although in the third modified example, the print device 1 includes the camera, the video may be picked up by a camera which is connectable with the print device 1 and is different from the image pickup unit 16.

In addition, although in one embodiment, the pulse wave information is acquired by analyzing the user's finger video, a way of acquiring the pulse wave information is not limited to analysis of the user's finger video. For example, pulse wave measurement may be either measurement which is performed by using a sensor that an ultraviolet ray light emitting element and a light receiving element are combined with each other or measurement which is performed by using a method of seizing a change in artery volume as a change in pressure by a pressure sensor.

In addition, in one embodiment, although the nail designs which are to be presented to the user are determined by downloading the nail designs which are stored in the nail design registration/distribution server to the user terminal 2, the way of acquiring the nail designs is not limited to the above. For example, the user terminal 2 may generate nail designs in accordance with the estimated psychosomatic state of the user. In addition, in one embodiment, although the nail designs which are presented to the user are displayed on the user terminal 2 so as to make the user select one of the nail designs, a way of selecting one nail design is not limited to the above. For example, a screen display function and a selection input function may be installed in the print device 1 and the print device 1 may display the nail designs which are presented on its own screen so as to make the user select one of the nail designs. In addition, in one embodiment, although the present invention is applied to the print device 1, an application object is not limited to the print device 1. For example, an information processing device which pertains to the present invention may be either the user terminal 2 or the server group 4. Although the print device 1 pertaining to one embodiment executes processing of detecting the video pulse wave and acquiring the biological information from the detected video pulse wave, and then determining the nail designs on the basis of the acquired biological information, in addition to printing of the nail designs and acquisition of the video of a part of the user's body, the processing of determining the nail designs may be executed on the user terminal 2 side and/or the server group 4 side.

That is, the nail printing system S may have a configuration which includes the print device 1 which has the image pickup unit 16 (the acquisition unit) which acquires the video of at least a part of the user's body, the communication unit 20 which transmits the video that the image pickup unit 16 acquires to the outside and the print unit 32 which prints the selected nail design on the user's nail 5 and the user terminal 2 which has the communication unit 59 which communicates with the print device 1 and the control unit 70 which determines the user's biological information on the basis of the video of at least one part of the user's body and determines the nail designs to be presented to the user on the basis of the determined biological information. In this configuration, the print unit 32 performs printing on the user's nail 5 on the basis of the nail design that the communication unit 20 receives from the communication unit 59. Incidentally, although in the above configuration, the print device 1 has the image pickup unit 16, the type of the image pickup unit 16 is not limited to the above-described one and an image pickup device which is installed separately from the print device 1 may be adopted as the image pickup unit 16 and the camera that the user terminal 2 has may be adopted as the image pickup unit 16. For example, in a case where the image pickup device which is installed separately from the print device 1 is adopted as the image pickup unit 16, the image pickup device which is adopted as the image pickup unit 16 acquires the video of at least a part of the user's body and then may transmit information on the video directly to either the user terminal 2 or the server group 4. In addition, in a case where the camera that the user terminal 2 has is adopted as the image pickup unit 16, the camera which is adopted as the image pickup unit 16 acquires the video of at least the part of the user's body and then either may transmit the information on the video directly to the server group 4 or may not transmit the information to the server group 4 and, instead, the control unit 110 may determine the nail design on the basis of information which relates to the video which is picked up by the camera that the user terminal 2 has as it is.

It is possible to execute the above-described series of processing in either hardware or software. In other words, the functional configuration in FIG. 4 is merely illustrative and there is no specific limitation on the configuration which executes the processing. That is, it is sufficient that the print device 1 be equipped with a function which makes it possible to execute the above-described series of processing as a whole. Which functional block is to be used for realizing this function is not particularly limited to the example in FIG. 4.

In addition, one function block may be configured by either a single piece of hardware or a single piece of software or may be configured by a combination of hardware and software. The functional configuration in one embodiment is realized by a processor which executes arithmetic processing. As processors which are usable in one embodiment, a processor which is configured by a single body of various kinds of processing devices such as a single processor, a multi-processor, a multi-core processor and so forth is included and, in addition, a processor that each of these various kinds of processing devices is combined with a processing circuit such as an ASIC (Application Specific Integrated Circuit), an FPGA (Field Programmable Gate Array) and so forth is also included.

In a case of executing the series of processing in software, a program which configures the software is installed into a computer and so forth over a network and from a recording medium. The computer may be also a computer which is incorporated into dedicated hardware. In addition, the computer may be a computer which becomes possible to execute various functions by installing various kinds of programs such as, for example, a general-purpose personal computer.

The recording medium which includes such a program as described above is configured not only by the removable media 100 in FIG. 2 which is distributed separately from the device main body in order in order to provide the program to the user but also by recording medium and so forth which are provided to the user in a state of being incorporated into the device main body in advance. The removable media 100 are configured by, for example, a magnetic disk (including a floppy disk), an optical disk, a magneto-optical disk and so forth. The optical disk is configured by, for example, a CD-ROM (Compact Disk-Read Only Memory), a DVD (Digital Versatile Disk), a Blue-ray (the registered trademark) Disc and so forth. The magneto-optical disk is configured by an MD (Mini-Disk) and so forth. In addition, the recording medium which is provided to the user in a state of being incorporated into the device main body in advance is configured by, for example, the ROM 12 in FIG. 2 that the program is recorded, a hard disk which is included in the storage unit 19 in FIG. 2 and so forth.

Incidentally, in the present specification, the steps which describe the program which is recorded into the recording medium include, of course, not only processes which are executed in a time series in order but also processes which are executed in parallel or individually, though not being necessarily processed in time series. In addition, in the present specification, the term “system” shall mean an overall apparatus which is configured by a plurality of devices, a plurality of units and so forth.

Although one embodiment of the present invention and several modified examples thereof are described above, the embodiment and modified examples thereof are merely illustrative and do not limit the technical scope of the present invention. It is possible for the present invention to assume other various embodiments and it is possible to make various alterations such as omission, substitution and so forth within a range not deviating from the gist of the present invention. One embodiment and the modified examples thereof are included in the scope and the gist of the present invention which is described in the present specification and so forth and are also included in the scope of the invention which is described in the appended claims and the scope of equivalents thereof.

What is claimed is:

1. An information processing device comprising:
at least one processor which acquires biological information which relates to a blood flow of a user and determines at least one nail design on the basis of the acquired biological information.

2. The information processing device according to claim 1, wherein
the processor acquires the biological information on the basis of information which relates to a video which is obtained by picking up an image of at least a part of the user's body.

3. The information processing device according to claim 2, wherein
the biological information is video-based photoplethysmography of the user.

4. The information processing device according to claim 2, wherein
the processor acquires the biological information on the basis of information which relates to a video which is obtained by picking up an image of a user's finger.

5. The information processing device according to claim 1, wherein
the processor decides a user's blood circulation state on the basis of the biological information and determines the nail designs on the basis of the decided user's blood circulation state.

6. The information processing device according to claim 1, wherein
the processor decides a user's nervous state on the basis of the biological information and determines the nail designs on the basis of the decided user's nervous state.

7. The information processing device according to claim 1, wherein
the processor decides a state of a user's heart rate adjustment function on the basis of the biological information and determines the nail designs on the basis of the decided user's heart rate adjustment function.

8. The information processing device according to claim 1, wherein
the processor decides a high/low state of a user's blood pressure on the basis of the biological information and determines the nail designs on the basis of the decided high/low state of the user's blood pressure.

9. The information processing device according to claim 1, wherein
groups which each are configured by a plurality of nail designs are set in advance, and
the processor determines one of the groups as the group of nail designs to be presented to the user on the basis of the biological information.

10. A nail printing system comprising:
a print device which has a first communication unit which communicates with the outside and a print unit which performs printing on a nail of a user; and
an information processing device which has a second communication unit which communicates with the print device and a process unit which determines biological information of the user on the basis of information which relates to a video of at least a part of the user's body and determines nail designs on the basis of the determined biological information, wherein
the print unit performs printing on the user's nail on the basis of the nail designs that the first communication unit receives from the second communication unit.

11. Computer readable media, making a computer execute:
a processing function of acquiring biological information which relates to a blood flow of a user; and
a processing function of determining nail designs to be presented to the user on the basis of the acquired biological information.

12. A nail design selection method that an information processing device executes comprising:
acquiring biological information which relates to a blood flow of a user; and
determining nail designs to be presented to the user on the basis of the acquired biological information.

13. The nail design selection method that the information processing device executes according to claim 12, further comprising:
acquiring the biological information on the basis of information which relates to a video which is obtained by picking up an image of at least a part of the user's body.

14. The nail design selection method that the information processing device executes according to claim 13, wherein
the biological information is video-based photoplethysmography of the user.

15. The nail design selection method that the information processing device executes according to claim 13, further comprising:
acquiring the biological information on the basis of information which relates to a video which is obtained by picking up an image of a user's finger.

16. The nail design selection method that the information processing device executes according to claim 12, further comprising:
deciding a user's blood circulation state on the basis of the biological information; and
determining the nail designs on the basis of the decided user's blood circulation state.

17. The nail design selection method that the information processing device executes according to claim 12, further comprising:
deciding a user's nervous state on the basis of the biological information; and
determining the nail designs on the basis of the decided user's nervous state.

18. The nail design selection method that the information processing device executes according to claim 12, further comprising:
deciding a state of a user's heart rate adjustment function on the basis of the biological information; and
determining the nail designs on the basis of the decided user's heart rate adjustment function.

19. The nail design selection method that the information processing device executes according to claim 12, further comprising:
deciding a high/low state of a user's blood pressure on the basis of the biological information; and
determining the nail designs on the basis of the decided high/low state of the user's blood pressure.

20. The nail design selection method that the information processing device executes according to claim 12, wherein
groups which each are configured by a plurality of nail designs are set in advance, and
further comprising:
determining one of the groups as the group of nail designs to be presented to the user on the basis of the biological information.
